(19) United States
(12) Patent Application Publication
(10) Pub. No.: US 2014/0039309 A1
(43) Pub. Date: Feb. 6, 2014

(54) VEIN IMAGING SYSTEMS AND METHODS

(71) Applicant: EVENA MEDICAL, INC., Los Altos, CA (US)

(72) Inventors: Melvyn L. Harris, Folsom, CA (US); Toni A. Harris, Folsom, CA (US); Frank J. Ball, Roseville, CA (US); David J. Gruebele, Folsom, CA (US); Ignacio E. Cespedes, Folsom, CA (US)

(73) Assignee: EVENA MEDICAL, INC., LOS ALTOS, CA (US)

(21) Appl. No.: 13/802,604

(22) Filed: Mar. 13, 2013

Related U.S. Application Data
(60) Provisional application No. 61/639,012, filed on Apr. 26, 2012.
(60) Provisional application No. 61/639,808, filed on Apr. 27, 2012.
(60) Provisional application No. 61/714,684, filed on Oct. 16, 2012.

(51) Int. Cl. A61B 5/00

(52) U.S. Cl. CPC A61B 5/0075
USPC 600/431; 600/473

(57) ABSTRACT

Some embodiments of this disclosure relates to systems and methods for imaging a patient's vasculature. For example, near infrared (NIR) light can be used to illuminate a target area and light that is reflected or scattered from the target area can be used for generate an image of the target area. In some embodiments, the system can be configured such that the image shows the presence, absence, or extent of infiltration or extravasation in the target area. The system can be configured to document that presence, absence, or extend of infiltration or extravasation at an infusion site. In some embodiments, an imaging system can be mounted onto a patient so that the imaging system can monitor an infusion site, and the imaging system can be configured to automatically detect the presence of infiltration or extravasation.

### CROSS-REFERENCE TO RELATED APPLICATIONS

This application claims the benefit under 35 U.S.C. §119(e) of U.S. Provisional Patent Application No. 61/639,012 (Attorney Docket No. EVENA.001PR), filed Apr. 26, 2012, and titled VEIN IMAGING SYSTEMS AND METHODS, U.S. Provisional Patent Application No. 61/639,808 (Attorney Docket No. EVENA.001PR2), filed Apr. 27, 2012, and titled VEIN IMAGING SYSTEMS AND METHODS, and U.S. Provisional Patent Application No. 61/714,684 (Attorney Docket No. EVENA.013PR), filed Oct. 16, 2012, and titled VEIN IMAGING SYSTEMS AND METHODS, each of which is hereby incorporated by reference in its entirety and made a part of this specification for all that it discloses.

### BACKGROUND

1. Field of the Disclosure

Some embodiments of this disclosure relate to systems and methods for imaging a patient's vasculature, such as to facilitate the insertion of an intravenous line or to facilitate assessment of a blood vessel, an infusion site, or a target area on a patient.

2. Description of the Related Art

Access to a patient's vasculature is typically obtained by advancing a needle through the patient's skin, subcutaneous tissue, and vessel wall, and into the lumen of a blood vessel. The exact location of the blood vessel may be difficult to determine because it is not in the direct sight of the medical practitioner attempting to gain vascular access. Placing the distal tip of the needle in the blood vessel lumen may also be difficult for similar reasons. Consequently, proper placement of hypodermic and procedural needles can be challenging.

Furthermore, because the patient's vasculature is not readily visible, it is often difficult for a medical practitioner to determine whether a patient's blood vessel has been compromised (e.g., due to vein collapse, vein blockage, vein leakage, etc.). If medical fluids are infused (e.g., via an IV connection) into a compromised blood vessel, the fluid can leak out of the blood vessel and into the surrounding tissue, resulting in infiltration or extravasation, which can cause damage to the surrounding tissue and can prevent infused medication from properly entering the patient's vasculature.

To check the patency of a blood vessel to determine whether the blood vessel is open and unobstructed, a medical practitioner generally infuses a fluid (e.g., saline) into the blood vessel (e.g., via an IV connection) and observes the area around the infusion site to determine whether the infiltration or extravasation has occurred. For example, the medical practitioner can feel the area around the infusion site to attempt to identify swelling, which can be an indication of infiltration or extravasation. In some cases, the area around the infusion site can bulge due to proper infusion of the fluid into a patent vein. Thus, it can be difficult for the medical practitioner to determine whether a blood vessel has been compromised, especially for low amounts of infiltration or extravasation. Also, in some instances, fluid can leak from an underside of the blood vessel (e.g., facing generally away from the surface of the skin), which can cause infiltration or extravasation that is relatively deep in the patient's tissue and is more difficult to detect using conventional patency checks.

### SUMMARY

Various embodiments disclosed herein can relate to a system for facilitating detection of infiltration or extravasation within a target area on a body portion of a patient. The system can include a light source configured to direct light onto the target area, a light sensor configured to receive light from the target area and to generate an image of the target area, and a display configured to display the image of the target area. The system can be configured such that the displayed image shows the presence of infiltration or extravasation when infiltration or extravasation is present in the target area.

The light source can be configured to emit near infrared (NIR) light. The light source can be configured to emit light between about 600 nm and about 1000 nm. The light source can be configured to emit light that is configured to be absorbed by oxygenated/deoxygenated hemoglobin such that the image is configured to distinguish between oxygenated/deoxygenated hemoglobin in blood and the surrounding tissue. The light source can be configured to emit light that is configured to be absorbed by oxygenated hemoglobin such that the image is configured to distinguish between oxygenated hemoglobin in blood and the surrounding tissue.

The system can be configured to facilitate an assessment of the patency of a vein, e.g., by providing an image that shows blood flow or an absence of blood flow when a medical practitioner strips the vein temporarily or when a medical practitioner infuses saline such that the saline is observable in the image as a displaced column moving through the vein. Various systems disclosed herein can be used for assessing blood flow in a vein as well as identifying infiltration and extravasation.

In some embodiments, the light sensor can be configured to receive light that is reflected or scattered from the target area. In some embodiments, the light source can be configured to be pulsed on and off at a rate that corresponds to an imaging rate of the light sensor.

The light source can include a first light emitter configured to emit light of a first wavelength, and a second light emitter configured to emit light of a second wavelength that is different than the first wavelength. The system can include a controller configured to pulse the first and second light emitters to produce a first image using the first wavelength of light and a second image using the second wavelength of light. The controller can be configured to display the first and second images in rapid succession so that the first and second images merge when viewed by a viewer. The controller can be configured to combine the first image and the second image to form a composite image for display. The light source can include a third light emitter configured to emit light of a third wavelength that is different than the first and second wavelengths, and the controller can be configured to pulse the third light emitter to produce a third image using the third wavelength. The first wavelength can be between about 700 nm and 800 nm, the second wavelength can be between about 800 nm and about 900 nm, and the third wavelength can be between about 900 nm to about 1100 nm. The light source can include a fourth light emitter configured to emit light of a fourth wavelength that is different than the first, second, and third wavelengths, and the controller can be configured to pulse the fourth light emitter to produce a fourth image using the fourth wavelength.

The system can include an optical filter disposed to filter light directed to the light sensor, the optical filter configured to attenuate light of at least some wavelengths not emitted by the light source. The system can include a camera that includes the light sensor. The camera can have at least one lens, and the optical filter can be disposed on a surface of the at least one lens.

In some embodiments, the system can include a controller configured to colorize the image.

For various different embodiments disclosed herein, the light sensor can include a first light sensor element configured to produce a right-eye image and a second light sensor element configured to produce a left-eye image, and the image can be a 3D stereoscopic image that includes the right-eye image and the left-eye image.

The system can be configured to display an image that shows the presence of infiltration or extravasation of at least about 3 mL to about 5 mL, at least about 1 mL to about 3 mL, and/or at least about 0.5 mL to about 1 mL. The system can be configured to display an image that shows the presence of infiltration or extravasation that is about 0.1 mm to about 3 mm deep in the tissue of the target area, that is about 3 mm to about 5 mm deep in the tissue of the target area, that is about 5 mm to about 7 mm deep in the tissue of the target area, and/or that is about 7 mm to about 10 mm deep in the tissue of the target area.

The system can include a controller configured to analyze the image to determine whether infiltration or extravasation is likely present based at least in part on the image, and display an indication on the display of whether infiltration or extravasation is likely present.

The system can include a controller configured to associate the image with a patient identifier and with time information and store the image and associated patient identifier and time information in a patient treatment archive. The controller can be configured to associate the image with a medical practitioner identifier, and store the associated medical practitioner identifier in the patient treatment archive. The controller can be configured to receive user input and store the image and associated metadata in the patient treatment archive in response to the user input. The system can include a patient treatment archive stored in a computer readable memory device in communication with the controller. The patient treatment archive can be searchable by the patient identifier. The patient identifier can include an image of a face of the patient. To associate the image with a patient identifier, the controller can be configured to store the image in an electronic folder or file associated with the patient.

The system can include a controller configured to receive medication information indicative of a medication to be administered to a patient, determine whether the medication to be administered to the patient is appropriate, based at least in part on the received medication information, and issue a warning if the medication to be administered to the patient is determined to be inappropriate or issue an approval if the medication to be administered to the patient is determined to be appropriate. To determine whether the medication to be delivered to the patient is appropriate, the controller can be configured to access one or more expected dosage values stored on a database, and compare a dosage value of the medication to be administered to the patient to the one or more expected dosage values. The controller can be configured to store the medication information in a patient treatment archive. The controller can be configured to store a patient identifier associated with the medication information in the patient treatment archive and store time information associated with the medication information in the patient treatment archive. The medication information can include an image of the medication to be delivered to the patient. The controller can be configured to receive a patient identifier and determine whether the medication to be administered to the patient is appropriate, based at least in part on the patient identifier.

Various embodiments disclosed herein can relate to a system for facilitating detection of infiltration or extravasation within a target area on a body portion of a patient, the system can include a light source configured to direct light onto the target area, a light sensor configured to receive light from the target area; and a controller configured to generate an image of the target area. The image can show the presence of infiltration or extravasation when infiltration or extravasation of at least between about 0.5 mL and about 5 mL is present in the target area.

The system can further include a display device that includes a screen for displaying the image. The light sensor can be configured to receive light reflected or scattered from the target area.

Various embodiments disclosed herein can relate to a method of imaging an infusion site on a patient to facilitate detection of infiltration or extravasation at the infusion site. The method can include illuminating the infusion site with light, receiving light from the infusion site onto a light sensor, generating an image of the infusion site from the light received by the light sensor, and displaying the image of the infusion site to a medical practitioner. The image can show the presence of infiltration or extravasation when infiltration or extravasation is present at the infusion site.

The method can included illuminating the infusion site with light comprises illuminating the infusion site with near infrared (NIR) light. In some embodiments, the light received by the light sensor is light reflected or scattered by the infusion site on the patient.

The image can show the absence of infiltration or extravasation when infiltration or extravasation is not present at the infusion site. The image can show the extent of infiltration or extravasation when infiltration or extravasation is present at the infusion site.

The method can include infusing an imaging enhancement agent through the infusion site. The imaging enhancement agent can include a biocompatible dye. The imaging enhancement agent can be a biocompatible near infrared fluorescent material. The imaging enhancement agent can include Indocyanine Green.

The method can include illuminating the infusion site with light of a first wavelength during a first time, and illuminating the infusion site with light of a second wavelength, different than the first wavelength, during a second time, different than the first time. Generating an image of the infusion site can include generating a first image using the light of the first wavelength and generating a second image using the light of the second wavelength. Displaying the image can include displaying the first image and the second image in rapid succession so that the first image and the second image merge when viewed by a viewer. Illuminating the infusion site can include illuminating the infusion site with light of a third wavelength that is different than the first and second wavelengths, and generating an image of the infusion site can include generating a third image using the light of the third wavelength.

The image can show the presence of infiltration or extravasation of at least about 3 mL to about 5 mL, of at least about 1 mL to about 3 mL, and/or at least about 0.5 mL to about 1 mL. The image can show the presence of infiltration or extravasation that is about 0.1 mm to about 3 mm deep in tissue at the infusion site, about 3 mm to about 5 mm dfeep in tissue at the infusion site, about 5 mm to about 7 mm in tissue at the infusion site, and/or about 7 mm to about 10 mm deep in tissue at the infusion site.

The method can include associating the image with a patient identifier and with time information and storing the image and associated patient identifier and time information in a patient treatment archive in a computer-readable memory device.

Various embodiments disclosed herein can relate to a method of facilitating assessment of an infusion site. The method can include illuminating the infusion site with light of a first wavelength, infusing an imaging enhancement agent into the infusion site, wherein the imaging enhancement agent is configured to absorb the light of the first wavelength and emit light of a second wavelength that is different than the first wavelength.

The imaging enhancement agent can be a biocompatible near infrared fluorescent (NIRF) material. The imaging enhancement agent can be at least one of NIRF dye molecules, NIRF quantum dots, NIRF single walled carbon nanotubes, and NIRF rare earth metal compounds. The imaging enhancement agent can be Indocyanine Green.

In some embodiments, the imaging enhancement agent can emit visible light. The method can include determining whether the vein is occluded based at least in part on the visible light emitted by the imaging enhancement agent. The method can include determining whether infiltration or extravasation is present at the infusion site based at least in part on the visible light emitted by the imaging enhancement agent.

The method can include receiving the light of the second wavelength onto a light sensor and generating an image of the infusion site from the light received by the light sensor.

Various embodiments disclosed herein can relate to a system for assessing an infusion site. The system can include an infusion device containing an imaging enhancement agent, and the infusing device can be configured to infuse the imaging enhancement agent into the infusion site. The system can include a light source, and the light source can be to emit light having a first wavelength onto the infusion site, and the imaging enhancement agent can be configured to absorb the light of the first wavelength and emit light of a second wavelength different than the first wavelength.

The imaging enhancement agent can be a biocompatible near infrared fluorescent (NIRF) material. The imaging enhancement agent can include at least one of NIRF dye molecules, NIRF quantum dots, NIRF single walled carbon nanotubes, and NIRF rare earth metal compounds. The imaging enhancement agent can include Indocyanine Green. In some embodiments, the imaging enhancement agent can emit visible light. The light source can be configured to emit near infrared (NIR) light. The system can include a light sensor configured to receiving the light of the second wavelength onto a light sensor, and a controller configured to generating an image of the infusion site from the light received by the light sensor.

Various embodiments disclosed herein can relate to a method of accessing a patient's vasculature. The method can include accessing an imaging device that includes a light source, a light sensor, and a controller. At a first time, the method can include illuminating a target area on a body portion of the patient with light from the light source on the imaging device, receiving light from the target area onto the light sensor of the imaging device, generating, using the controller, a first image of the target area from the light received by the light sensor. The first image can be configured to distinguish between one or more vein in the target area and other tissue surrounding the veins in the target area, such that the image is configured to facilitate insertion of an intravenous line to establish an infusion site. At a second time that is later than the first time, the method can include imaging the infusion site using the imaging device to facilitate detection of infiltration or extravasation at the infusion site as recited herein.

The method can include inserting an intravenous line into the target area to establish an infusion site, and the first image can be used to facilitate the insertion of the intravenous line.

Various embodiments disclosed herein can relate to a method of documenting the presence and/or absence of infiltration or extravasation for infusion sites of patients. The method can include storing a plurality of images in a patient treatment archive on a computer-readable memory device. The plurality of images can be of infusion sites on a plurality of patients, and the plurality of images can be configured to show the presence of infiltration or extravasation when infiltration or extravasation was present at the infusion site. The method can include storing patient identifiers associated with the plurality of images, storing time information associated with the plurality of images, and retrieving, using one or more computer processors in communication with the computer-readable memory device, one or more images of an infusion site on the particular patient from the patient treatment archive.

The method can include receiving a notification of a claim of medical error for a particular patient. The claim of medical error can include at least one of a law suit, an insurance claim, an allegation, a patient complaint, a threat of legal action, a co-worker complaint, and a criminal charge or investigation. The method can include using the one or more images to confirm a presence or absence of infiltration or extravasation at an infusion site on the particular patient at a particular time. The plurality of images can be produced using near infrared (NIR) light.

The method can include, for each of the plurality of images, illuminating the infusion site with light, receiving light from the infusion site onto a light sensor, generating the image of the infusion site from the light received by the light sensor, and displaying the image of the infusion site to a medical practitioner.

The method can include storing, using the one or more computer processors, medical practitioner identifiers associated with the plurality of images. The patient identifiers can include images of the faces of the plurality of patients. The patient identifiers can include electronic folders or files associated with the plurality of patients.

The method can include storing, in the patient treatment archive, medication information indicative of medication administered to the plurality of patients, and retrieving, using the one or more computer processors, medication information indicative of medication delivered to the particular patient from the patient treatment archive.

Various embodiments disclosed herein can relate to a system for documenting the presence and/or absence of infiltration or extravasation for infusion sites on patients. The system can include a patient treatment archive stored in a computer-readable memory device, and the patient treatment archive can include a plurality of images of infusion sites on a plurality of patients, where the plurality of images can show the presence of infiltration or extravasation when infiltration or extravasation is present at the infusion site. The patient treatment archive can include a plurality of patient identifiers associated with the plurality of images and time information associated with the plurality of images. A controller comprising one or more computer processors in communication with the computer-readable memory device, can be configured to retrieve one or more images from the patient treatment archive based at least in part on a specified patient identifier.

The plurality of images can be produced using near infrared (NIR) light. The patient treatment archive can include comprises medical practitioner identifiers associated with the plurality of images.

The system can include a unit for facilitating detection of infiltration or extravasation at infusion sites on the plurality of patients, and the unit can include a light source configured to direct light onto the infusion sites, a light sensor configured to receive light from the infusion sites and to generate the images of the infusion sites, and a display configured to display the images of the infusion sites.

The patient identifiers can include images of the faces of the plurality of patients. The patient identifiers can include electronic folders or files associated with the plurality of patients.

The patient treatment archive can include medication information indicative of medication administered to the plurality of patients, and the controller can be configured to retrieve medication information indicative of delivered medication based at least in part on the specific patient identifier.

Various embodiments disclosed herein can relate to a non-transitory computer-readable medium device comprising computer-executable instructions configured to cause one or more computer processors to receive a plurality of images of infusion sites on a plurality of patients, where the images can be configured to show the presence of infiltration or extravasation when infiltration or extravasation was present at the infusion sites and/or the absence of infiltration or extravasation when infiltration or extravasation was not present at the infusion sites, store the plurality of images in a patient treatment archive, where each of the plurality of images is associated with a patient identifier, and retrieve one or more images from the patient treatment archive based at least in part on a specified patient identifier.

The computer-executable instructions can be configured to cause the one or more computer processors to provide a user interface configured to receive the specified patient identifier. The computer-executable instructions can be configured to cause the one or more computer processors to receive patient identifiers and to associate the patient identifiers with the plurality of images.

Each of the plurality of images can be associated with a medical practitioner identifier. The computer-executable instructions can be configured to cause the one or more computer processors to receive medical practitioner identifiers and associate the medical practitioner identifiers with the plurality of images. Each of the plurality of images can be associated with time information.

The patient identifier can be an electronic folder or file associate with a patient. The patient identifiers can be associated with the images as metadata. The patient identifiers can be incorporated into headers of image files for the images.

The computer-executable instructions can be configured to cause the one or more computer processors to receive medication information indicative of medication administered to the plurality of patients, store the medication information in the patient treatment archive, where the medication information is associated with patient identifiers, and retrieve medication information indicative of delivered medication based on the specified patient identifier.

Various embodiments disclosed herein can relate to a system that includes a light source configured to direct light onto a target area on a patient, where the target area comprises one or more veins and other tissue around the veins, a light sensor configured to receive light from the target area, and a controller configured to operate the light source and light sensor to generate an image of the target area that is configured to distinguish the one or more veins from the other tissue around the veins. The controller can be configured to receive a patient identifier associated with the patient.

The system can be configured to determine whether a medical procedure is appropriate for the patient based at least in part on the patient identifier. The medical procedure can include inserting an intravenous line. The medical procedure can include administering a medication.

Various embodiments disclosed herein can relate to a system for providing information to a remote medical practitioner. The system can include a light source configured to direct non-visible light onto a target area, at least one light sensor configured to receive the non-visible light from the target area and generate a first image of the target area using the non-visible light. The at least one light sensor can be configured to receive visible light and generate a second image of the target area using visible light. The system can include a communication interface configured to transmit the second image to a remote system accessible to the remote medical practitioner.

The at least one light sensor can include a first light sensor configured to generate the first image using the non-visible light and a second light sensor configured to generate the second image using visible light.

The system can include one or more medical components configured to obtain information relating to one or more patient conditions, and the communication link can be configured to transmit the information obtained from the one or more medical components to the remote system. The one or more medical components can include one or more of a pulse oximeter, an ultrasound device, an ECG/EKG device, a blood pressure monitor, a digital stethoscope, a thermometer, an otoscope, or an exam camera.

The system can include an audio sensor configured to produce a signal from sound received by the audio sensor, and the communication interface can be configured to transmit the signal to the remote system.

The light source and the at least one light detector can be incorporated onto a wearable system. The wearable system can be a head mountable display system configured to display information to a wearer. The head mountable display system can include a display configured to be disposed in front of a wearer's eye when worn. The head mountable display system can include a right display configured to be disposed in front of a wearer's right eye, a left display configured to be disposed in front of a wearer's left eye, and the at least one light sensor can include a right sensor configured to produce a right-eye image and a left sensor configured to produce a left-eye image, and the right-eye image and the left-eye image can be configured to produce a stereoscopic 3D image of the target area. The system can be configured to produce the stereoscopic 3D image using non-visible light. The system can be configured to produce the stereoscopic 3D image using near infrared (NIR) light. The system can be configured to produce the stereoscopic 3D image using visible light.

The communication interface can be configured to receive information from the remote system, and the system can be configured to present the information using an output device. The output device can include a display. The information can include audio information and the output device can include an audio output device. The communication interface can be configured to receive medical treatment instructions from the remote system.

The system can be configured such that the first image is configured to distinguish one or more veins in the target area from other body tissue in the target area. The system can further include a display configured to display the first image, and the non-visible light can be configured to be reflected or scattered less by blood in one or more veins in the target area than by other body tissue in the target area.

The non-visible light can be configured to be absorbed by oxygenated/deoxygenated hemoglobin such that the first image is configured to distinguish between oxygenated/deoxygenated hemoglobin in blood and the surrounding tissue. The non-visible light can be configured to be absorbed by oxygenated hemoglobin such that the first image is configured to distinguish between oxygenated hemoglobin in blood and the surrounding tissue. The non-visible light can include near infrared (NIR) light.

Various embodiments disclosed herein can relate to a system that includes a light source configured to direct light onto a target area that comprises one or more veins and other tissue around the veins, a light sensor configured to receive light from the target area, a controller configured to operate the light source and light sensor to generate an image of the target area that is configured to distinguish the one or more veins from the other tissue around the veins, and one or more medical components configured to provide information relating to one or more patient conditions. The controller can be configured to receive the information from the one or more medical components. The light sensor can be configured to receive light reflected or scattered from the target area, in some embodiments.

The one or more medical components comprise one or more of a pulse oximeter, an ultrasound device, an ECG/EKG device, blood pressure monitor, a digital stethoscope, a thermometer, an otoscope, or an exam camera.

The system can include a communication interface configured to transmit the information received from the one or more medical components to a remote system accessible to the remote medical practitioner. The system cam further include an audio sensor configured to produce a signal from sound received by the audio sensor, and the communication interface can be configured to transmit the signal to the remote system. The communication interface can be configured to receive information from the remote system, and the system can be configured to present the information using an output device. The information can include audio information and the output device can include an audio output device. The output device can include a display. The communication interface can be configured to receive medical treatment instructions from the remote system.

The light is configured to be reflected or scattered less by blood in the one or more veins than by the other tissue in the target area. The light can include near infrared (NIR) light. The system can include a display configured to display the image of the target area, and the display can be configured to display the information received from the one or more medical components.

The system can include a patient integration module that can be configured to receive a plurality of cables for a plurality of medical components configured to provide information relating to a plurality of patient conditions, and the patient integration module can be configured to provide a single cable configured to transmit the information received from the plurality of medical components to the controller.

The light source and the light sensor can be incorporated onto a wearable system. The wearable system can be a head mountable display system configured to display information to a wearer. The head mountable display system can include a display, which can be configured to be disposed in front of a wearer's eye when worn. The head mountable display system can include a right display configured to be disposed in front of a wearer's right eye, and a left display configured to be disposed in front of a wearer's left eye, and the light sensor can include a right sensor configured to produce a right-eye image and a left sensor configured to produce a left-eye image, and the controller can be configured to generate a stereoscopic 3D image of the target area.

Various embodiments disclosed herein can relate to a method of treating a patient, and the method can include illuminating a body portion of the patient with light from a light source on a wearable system, where the body portion comprises one or more vein and other body tissue around the veins, receiving light from the body portion onto a light sensor on the wearable system, generating an image from the light received by the light sensor, where the image is configured to distinguish between the one or more veins and the other body tissue around the veins, such that the image is configured to facilitate insertion of an intravenous line into one of the one or more veins. The method can include receiving information from one or more medical components, the information relating to one or more patient conditions, and transmitting, using a communication interface on the wearable system, the information from the one or more medical components to a remote system that is accessible to a medical practitioner.

The wearable system can be worn by a local medical practitioner at the patient's location. The method can include inserting an intravenous line into one of the one or more veins using the image to facilitate the insertion of the intravenous line. The method can include operating the one or more medical components to collect the information relating to one or more patient conditions. The method can include receiving patient treatment information from the remote system. The method can include treating the patient based at least in part on the treatment information received from the remote system. Treating the patient can include infusing a treatment fluid through the intravenous line.

The wearable system can include first and second cameras and generating an image can include generated a stereoscopic 3D image of the body portion.

The method can include transmitting, via the communication interface, audio information to the remote system. The method can include receiving, via the communication interface, audio information from the remote system.

Various embodiments disclosed herein can relate to a system for providing stereoscopic 3D viewing of a patient's vasculature in a target area, the system can include a light source configured to direct light onto the target area, a first light sensor positioned at a location configured to not be coincident with a normal line of sight of a user's eye. The first light sensor can be configured to receive light from the target area to generate a right-eye image of the target area. The system can include a second light sensor spaced apart from the first light sensor and positioned at a location configured to not be coincident with a normal line of sight of the user's eye. The second light sensor can be configured to receive light from the target area to generate a left-eye image of the target area. A display module can be configured to present the right-eye and left-eye images to the user to provide stereoscopic 3D viewing of the patient's vasculature, wherein the right-eye and left-eye images can be configured to distinguish one or more veins in the target area from surrounding body tissue in the target area.

The display module can include a head-mounted display system that includes a right-eye display configured to display the right-eye image and a left-eye display configured to display the left-eye image. One or both of the first light sensor and the second light sensor can be disposed at temple regions of the head-mounted display system. The light can be configured to be reflected or scattered less by blood in one or more veins in the target area than by other tissue in the target area.

Various embodiments disclosed herein can relate to a system for viewing a patient's vasculature in a target area, can the system can include a wearable member configured to be worn by a user, a movable member that is movable with respect to the wearable member, wherein the movable member can be movable between a deployed position and a neutral position. The system can include a light source on the movable member, and the light source can be configured to direct light onto the target area when the movable member is in the deployed position. The system can include a light sensor on the movable member, and the light sensor can be configured to receive light from the target area when the movable member is in the deployed position. The system can include a controller configured to operate the light source and light sensor to generate an image of the target area that is configured to distinguish the one or more veins from the other tissue around the veins.

The wearable member can include a strap. The wearable member can be configured to be worn on a forearm of the user. The wearable member can be configured to be worn around the neck of the user. The movable member can be configured to pivot with respect to the movable member. The system can include a main body coupled to the wearable member, and the movable member can be movable with respect to the main body.

The main body can include a display configured to display the image. The light sensor can be covered when the movable member is in the neutral position. The system can include a connection portion that is configured to bias the movable member to one or both of the deployed position and the neutral position. The movable member can be configured to align with the user's forearm when in the neutral position, and the movable member can be configured to extend past an edge of the user's forearm when in the deployed position such that light from the light source can direct light past the user's forearm to the target area and such that the light sensor can receive light from the target area.

The system can include an attachment portion that is configured to receive a mobile device, and the attachment portion can have a communication interface element configured to establish a communication link between the mobile device and the light sensor when the mobile device is attached to the attachment portion.

The light source is configured to emit near infrared (NIR) light. The light sensor can be configured to receive light reflected or scattered by the target area when the movable member is in the deployed position.

Various embodiments disclosed herein can relate to a method of assessing the patency of a vein at an infusion site on a patient. The method can include infusing an infusion fluid into the vein through the infusion site, illuminating the infusion site area with light, receiving light from infusion site onto a light sensor, generating an image of the infusion site from the light received by the sensor, where the image can be configured to distinguish between blood in the vein and the infusion fluid in the vein, and determining whether the vein is occluded based at least in part on the image of the infusion site.

Determining whether the vein is occluded can be performed automatically by a controller that includes one or more computer processors.

Various embodiments disclosed herein can relate to a system for assessing the patency of a vein at an infusion site on a patient. The system can include a light source configured to illuminate the infusion site area with light, a light sensor configured to receiving light from infusion site onto a light sensor to produce image data of the infusion site, where the image data can be configured to distinguish between blood in the vein and an infusion fluid in the vein, and a controller configured to analyze the image data and automatically determine whether the vein is likely occluded based at least in part on the image data.

Various embodiment disclosed herein can relate to a system for viewing a patient's vasculature. The system can include a light source configured to direct light onto a target area that includes one or more veins and other tissue around the veins, a light sensor configured to receive light from the target area, and a controller configured to pulse the light at a rate that corresponds to an imaging rate of the light source and to generate an image of the target area from the light received by the light sensor. The image can be configured to distinguish the one or more veins from the other tissue around the veins.

Various embodiments disclosed herein can relate to a system for viewing a patient's vasculature. The system can include a light source configured to direct light onto a target area that comprises one or more veins and other tissue around the veins. The light source can include a first light emitter configured to emit light of a first wavelength, a second light emitter configured to emit light of a second wavelength that is different than the first wavelength. The system can include a light sensor configured to receive light from the target area and a controller configured to generate an image of the target area from the light received by the light sensor. The image can be configured to distinguish the one or more veins from the other tissue around the veins.

The controller can be configured to pulse the first and second light emitters to produce a first image using the first wavelength of light and a second image using the second wavelength of light. The controller can be configured to display the first and second images in rapid succession so that the first and second images merge when viewed by a viewer. The light source can include a third light emitter configured to emit light of a third wavelength that is different than the first and second wavelengths, and the controller can be configured to pulse the third light emitter to produce a third image using the third wavelength. The first wavelength can be between about 700 nm and 800 nm, and the second wavelength is between about 800 nm and about 900 nm, and the third wavelength can be between about 900 nm to about 1100 nm. The light source can include a fourth light emitter configured to emit light of a fourth wavelength that is different than the first, second, and third wavelengths, and the controller can be configured to pulse the fourth light emitter to produce a fourth image using the fourth wavelength. The first wavelength can be between about 700 nm and 775 nm, and the second wavelength can be between about 775 nm and about 825 nm, and the third wavelength can be between about 825 nm to about 875 nm, and the fourth wavelength can be between about 875 nm to about 1000 nm.

Various embodiments disclosed herein can relate to a system for viewing a patient's vasculature. The system can include a light source configured to direct light onto a target area that comprises one or more veins and other tissue around the veins, a digital light sensor configured to receive light from the target area, a controller configured to generate an image of the target area from the light received by the light sensor. The controller can be configured to perform digital image processing on enhance the image, and the image can be configured to distinguish the one or more veins from the other tissue around the veins.

The system can include a digital display configured to display the image. The system can further include a digital communication link between the digital display and the controller. The system can include a digital-format cable coupling the digital display to the controller. The controller can be configured to perform digital pre-processing on the image. The controller can be configured to perform digital post-processing on the image.

Various embodiments disclosed herein can relate to a system for viewing a patient's vasculature. The system can include a light source configured to direct light onto a target area that comprises one or more veins and other tissue around the veins, a light sensor configured to receive light from the target area, where the light sensor can include a first light sensor element configured to produce a right-eye image and a second light sensor element configured to produce a left-eye image, a controller configured to generate a 3D stereoscopic image that comprises the right-eye image and the left-eye image. The 3D stereoscopic image configured to distinguish the one or more veins from the other tissue around the veins. The system can include a display having a single screen for displaying the right-eye image and the left-eye image.

Various embodiments disclosed herein can relate to a system for monitoring an infusion site on a body portion of a patient. The system can include a light source, a light sensor, a support member configured to position the light source and the light sensor relative to the body portion of the patient such that light from the light source is directed onto the infusion site, and such that the light sensor receives light from the infusion site to generate image data of the infusion site, and a controller configured to analyze the image data and automatically detect the presence of infiltration or extravasation based at least in part on the image data.

The controller can be configured to send an instruction to an infusion pump to stop infusion in response to the detection of infiltration or extravasation. The controller can be configured to post an alarm upon detection of infiltration or extravasation.

The system can include a communication interface configured to send the image data from the light sensor to the controller. The controller can be located on an imaging head that includes the light source and the light sensor.

The controller can be configured to automatically detect, based at least in part on the image data, at least infiltration or extravasation of about 3 mL to about 5 mL, or of about 1 mL to about 3 mL, or of about 0.5 mL to about 1 mL. The controller can be configured to automatically detect, based at least in part on the image data, at least infiltration or extravasation that is about 0.1 mm to about 3 mm deep in tissue of the infusion site, that is about 1 mm to about 3 mm deep in tissue of the infusion site, that is about 3 mm to about 5 mm deep in tissue of the infusion site, that is about 5 mm to about 7 mm deep in tissue of the infusion site, and/or that is about 7 mm to about 10 mm deep in tissue of the infusion site.

The support member can be configured to position the light sensor relative to the infusion site to image an area of about three square inches to about five square inches, an area of about one square inch to about three square inches, and/or an area of about 0.1 square inches to about one square inch. The support member can be configured to couple the light sensor to the body portion of the patient.

The system can include a supporting portion configured to be positioned generally adjacent the infusion site and an extension portion configured to extend from the supporting portion such that at least a portion of the extension portion is positioned generally over the infusion site. The light source and the light sensor can be positioned in or on the supporting portion. The system can include one or more light guides configured to guide light from the extension portion to the light source and to guide line from the light source to the extension portion. In some embodiments, the light source and the light sensor can be positioned on the extension portion such that the light source and light sensor can be configured to be disposed generally over the infusion site.

The system can include an imaging head that comprises the light sensor and the light source, and at least a portion of the imaging head can be removably attachable to at least a portion of the support member. The at least a portion of the support member can be disposable, and the at least a portion of the imaging head can be configured to be reusable.

The support member can include a generally dome-shaped structure configured to suspend the light source and the light sensor over the infusion site. The dome-shaped structure can include a material that is substantially transparent to visible light to allow a medical practitioner to view the infusion site through the dome-shaped structure. The dome-shaped structure can include openings for providing ventilation between the infusion site and the area outside the dome-shaped structure.

The support member can include a strap configured to engage the body portion of the patient. The support member can include a flange configured to receive an adhesive for coupling the support member to the body portion of the patient.

The light source can be configured to emit near infrared (NIR) light. The support member can be configured to position the light sensor to receive light that is reflected or scattered by the infusion site.

The system can be configured to automatically generate image data of the infusion site and detect whether infiltration or extravasation is present at least about once every 1 minute to 5 minutes, at least about once every 10 seconds to 1 minute, or at least about once every 1 second to 10 seconds. The system can be configured to monitor the infusion site on a substantially continuous basis. The controller can be configured to receive a user input and adjust how often the controller generates image data of the infusion site and detects whether infiltration or extravasation is present based at least in part on the user input.

The system can include a display configured to display an image of the infusion site based on the image data. The controller can be configured to send the image data to a display in response to detection of infiltration or extravasation.

The controller can be configured to perform image processing on the image data to detect the presence of infiltration or extravasation. The controller can be configured to compare the image data to a baseline image to detect the presence of infiltration or extravasation. The controller can be configured to detect the presence of infiltration or extravasation based at least in part on a rate of change of the brightness or darkness of at least a portion of the image data.

The controller can be configured to associate the image data with a patient identifier and with time information, and store the image data and the associated patient identifier and time information in a patient treatment archive.

Various embodiments disclosed herein can relate to a system for monitoring a target area on a body portion of a patient. The system can include a light source, a light sensor, a communication interface; a support member configured to position the light source and the light sensor relative the target area such that light from the light source is directed onto the target area, and such that the light sensor receives light from the target area to generate image data of the target area. The image data can be capable of showing the presence of infiltration or extravasation in the target area. A communication interface can be configured to send the image data of the body portion to a controller.

The support member can be configured to position the light sensor relative to the infusion site to image an area of about three square inches to about five square inches, an area of about one square inch to about three square inches, or an area of about 0.1 square inches to about one square inch. The support member can be configured to couple the light sensor to the body portion of the patient.

The support member can include a generally dome-shaped structure configured to suspend the light source and the light sensor over the infusion site. The dome-shaped structure can include a material that is substantially transparent to visible light to allow a medical practitioner to view the infusion site through the dome-shaped structure. The dome-shaped structure can include openings for providing ventilation between the infusion site and the area outside the dome-shaped structure.

The system can include a supporting portion configured to be positioned generally adjacent the infusion site and an extension portion configured to extend from the supporting portion such that at least a portion of the extension portion is positioned generally over the infusion site. The light source and the light sensor can be positioned in or on the supporting portion. The system can include one or more light guides configured to guide light from the extension portion to the light source and to guide line from the light source to the extension portion. The light sensor and the light source can be positioned on the extension portion such that the light source and light sensor are configured to be disposed generally over the infusion site.

The system can include an imaging head that includes the light source and the light sensor, and at least a portion of the imaging head can be removably attachable to at least a portion of the support member. The at least a portion of the support member can be disposable, and the at least a portion of the imaging head can be configured to be reusable.

The support member can include a strap configured to engage the body portion of the patient. The support member can include a flange configured to receive an adhesive for coupling the support member to the body portion of the patient.

The light source can be configured to emit near infrared (NIR) light. The support member can be configured to position the light sensor to receive light that is reflected or scattered by the infusion site.

Various embodiments disclosed herein can relate to a method of infusing a medical fluid into a patient. The method can include actuating an infusion pump to infuse a medical fluid into a patient through an infusion site located on a body portion of the patient, illuminating the body portion with light, receiving light from the body portion onto a light sensor, generating image data from the light received by the light sensor, analyzing the image data using a controller that comprises one or more computer processors to automatically detect the presence of infiltration or extravasation, and automatically stopping the infusion pump to cease infusion of the medical fluid in response to a detection of infiltration or extravasation. In some embodiments, the method can include automatically posting an alarm, using the controller, in response to the detection of infiltration or extravasation.

The controller can be configured to automatically detect, based at least in part on the image data, infiltration or extravasation of about 3 mL to about 5 mL, of about 1 mL to about 3 mL, and/or of about 0.5 mL to about 1 mL.

The controller can be configured to automatically detect, based at least in part on the image data, infiltration or extravasation that is about 0.1 mm to about 3 mm deep in tissue of the infusion site, about 3 mm to about 5 mm deep in tissue of the infusion site, about 5 mm to about 7 mm deep in tissue of the infusion site, or about 7 mm to about 10 mm deep in tissue of the infusion site.

The method can include positioning the light sensor relative to the infusion site to image an area of about three square inches to about five square inches, to image an area of about one square inch to about three square inches, or to image an area of about 0.1 square inches to about one square inch. The method can include coupling the light sensor and the light source to the body portion of the patient using a support member.

The light can be near infrared (NIR) light. The light sensor can receive light that is reflected or scattered by the infusion site.

The method can include automatically generating image data of the infusion site and automatically detecting whether infiltration or extravasation is present at least about once every 1 minute to 5 minutes, at least about once every 10 seconds to 1 minute, or at least about once every 1 second to 10 seconds. The method can include monitoring the infusion site on a substantially continuous basis.

The method can include sending the image data to a display in response to detection of infiltration or extravasation.

Analyzing the image data can include performing image processing on the image data using the controller to detect the presence of infiltration or extravasation. Analyzing the image data can include comparing the image data to a baseline image to detect the presence of infiltration or extravasation. Analyzing the image data can include analyzing a rate of change of the brightness or darkness of at least a portion of the image data.

The method can include associating the image data with a patient identifier and with time information, and storing the image data and the associated patient identifier and time information in a patient treatment archive in a computer-readable memory device.

Various embodiments disclosed herein can relate to a method of automatically detecting infiltration or extravasation. The method can include receiving a signal from a light sensor, generating image data from the signal received from the light sensor, analyzing the image data using a controller that comprises one or more computer processors to automatically detect the presence of infiltration or extravasation based at least in part on the image data. In some embodiments, The method can include automatically posting an alarm, using the controller, in response to the detection of infiltration or extravasation.

The controller can be configured to automatically detect, based at least in part on the image data, infiltration or extravasation of about 3 mL to about 5 mL, of about 1 mL to about 3 mL, and/or of about 0.5 mL to about 1 mL.

The controller can be configured to automatically detect, based at least in part on the image data, infiltration or extravasation that is about 0.1 mm to about 3 mm deep in tissue of the infusion site, about 3 mm to about 5 mm deep in tissue of the infusion site, about 5 mm to about 7 mm deep in tissue of the infusion site, about 7 mm to about 10 mm deep in tissue of the infusion site.

The light sensor can be configured to generate the signal responsive to near infrared (NIR) light. The method can include sending the image data to a display in response to detection of infiltration or extravasation.

Analyzing the image data can include performing image processing on the image data using the controller to detect the presence of infiltration or extravasation. Analyzing the image data can include comparing the image data to a baseline image to detect the presence of infiltration or extravasation. Analyzing the image data can include analyzing a rate of change of the brightness or darkness of at least a portion of the image data.

The method can include associating the image data with a patient identifier and with time information, and storing the image data and the associated patient identifier and time information in a patient treatment archive in a computer-readable memory device.

### BRIEF DESCRIPTION OF THE DRAWINGS

FIG. 1 shows an example embodiment of an imaging system that can be used to generate images of veins or other vessels in body tissue of a patient.

FIG. 2 shows the imaging system of FIG. 1 displaying an image that shows the presence of infiltration or extravasation in a patient's body tissue.

FIG. 3 is an example plot of the optical output from a light source that includes three light emitters.

FIG. 4 shows an example embodiment of an imaging system incorporated into an integrated device.

FIG. 5 shows the device of FIG. 4 coupled to an articulated arm and mounted onto a vertical support.

FIG. 6 shows the device of FIG. 4 coupled to a point of care cart.

FIG. 7A shows the device of FIG. 4 coupled to a clinic utility cart.

FIG. 7B shows an example embodiment of a hand-held device incorporating the imaging system.

FIG. 8 schematically shows a system for document patient treatment, which can utilize the imaging system to generate and store images that document patency checks performed on multiple patients.

FIG. 9 includes flowcharts for example embodiments of methods relating to visualizing a site on a patient, inserting an intravenous (IV) line, documenting the insertion of the IV line, periodically flushing the IV line, and documenting the periodic flushing of the IV line.

FIG. 10 shows an example embodiment of a system for confirming medication to be administered to a patient.

FIG. 11 shows an example embodiment of an imaging system incorporated into an eyeglass.

FIG. 12 shows an example embodiment of an imaging system incorporated into a headband.

FIG. 13 shows an example embodiment of an imaging system incorporated into a helmet.

FIG. 14 shows an example embodiment of a 3D imaging system incorporated into an eyeglass.

FIG. 15 is a schematic diagram of certain components of an example embodiment of an imaging system.

FIG. 16 is a schematic diagram of certain components of another example embodiment of an imaging system.

FIG. 17 is a schematic diagram of multiple medical components in communication with a processor of an imaging system.

FIG. 18 is a schematic diagram of multiple medical components in communication with a patient integration module that is in communication with the processor of an imaging system.

FIG. 19 is a schematic diagram of a system for transferring medical information to facilitate on-site treatment of a patient.

FIG. 20 shows an example embodiment of an imaging system configured to be worn by a user.

FIG. 21 shows a movable portion of the imaging system of FIG. 20.

FIG. 22A shows the imaging system of FIG. 20 in a deployed configuration.

FIG. 22B shows another example embodiment of an imaging system that is configured to be worn by a user.

FIG. 22C shows the imaging system of FIG. 22B in a deployed configuration.

FIG. 22D shows an imaging system that is wearable by a user in an intermediate configuration.

FIG. 22E shows another example embodiment of an imaging system that is configured to be worn by a user.

FIG. 22F shows another example embodiment of an imaging system that is configured to be worn by a user.

FIG. 23 is a schematic diagram of an example embodiment of an imaging system for monitoring an infusion site.

FIG. 24 shows an example embodiment of a support member for the system of FIG. 23.

FIG. 25 shows another example embodiment of a support member for the system of FIG. 23.

FIG. 26 shows another example embodiment of a support member for the system of FIG. 23.

FIG. 27 shows another example embodiment of an imaging system for monitoring an infusion site.

FIG. 28 shows another example embodiment of an imaging system for monitoring an infusion site.

### DETAILED DESCRIPTION OF CERTAIN EMBODIMENTS

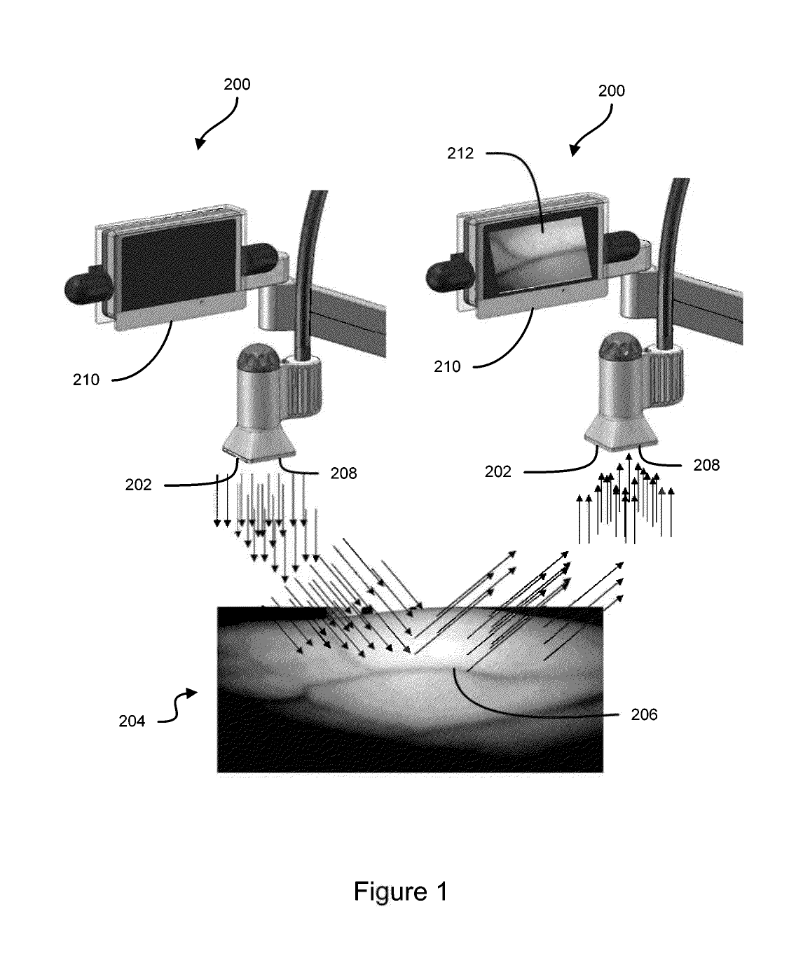

FIG. 1 shows an imaging system 200 that can be used to view the patent's vasculature and/or to identify infiltration or extravasation. The system can include a light source 202, such as an array of light emitting diodes (LEDs), that is configured to emit light onto a target area 204, such as an arm or other portion of a patient's body that includes one or more blood vessels 206 (e.g., veins). Although various embodiments are described herein with connection to viewing of veins, various features and methods described herein can be used for imaging other blood vessels and for imaging other vessels (e.g., lymphatic vessels) that transfer bodily fluids other than blood. The light source 202 can emit wavelengths of light that cause less of the light to be reflected or scattered by the veins and more of the light to be reflected by the tissue surrounding the veins. As used herein, the term “reflected” light includes light that is scattered. For example, near infrared (NIR) light can be used, e.g., having wavelengths between about 700 nm and about 1000 nm, although other wavelengths of light can be used. For example, in some embodiments, light of wavelengths between about 600 nm and 1100 nm can be used, and other wavelengths outside these ranges can be used in some instances. In some embodiments, light having wavelengths between about 800 nm and about 950 nm can be emitted by the light source 202. The NIR light can be generally absorbed by the hemoglobin in the blood in the veins, and the NIR light can be generally reflected or scattered by the subcutaneous tissue around the veins. In some embodiments, the light from the light source 202 can have a wavelength such that the light is absorbed by de-oxygenated and/or oxygenated hemoglobin in the blood such that the imaging system 200 is able to distinguish between the de-oxygenated and/or oxygenated hemoglobin in blood (e.g., inside a vein 206) and body tissue surrounding the vein 206. By using wavelengths of light that have significant absorption for both de-oxygenated and oxygenated hemoglobin, the imaging system 200 can provide for improved imaging of the veins 206 or of blood located outside of a vein 206 (e.g., in the case of infiltration or extravasation).

The system can include a light sensor 208 (e.g., a camera) that is sensitive to the one or more wavelengths of light emitted by the light source so that the light sensor can generate an image of the target area in which the veins are visible. A display device, such as a display 210 having a screen, can display the image 212 generated by the light sensor 208. In some embodiments, the system can include multiple displays 210, and the imaging head 214 (which can be a handheld device) can be configured to send the image to multiple different displays. The imaging head 214 can be battery powered and can be configured to transmit images wirelessly, or a cable can be used to deliver power to the imaging head and/or to transmit image signals from the imaging head 214. In some embodiments, the system can include a printer for printing the image generated by the light sensor, and the printer can be included in addition to the display 210, or in place thereof. As shown in FIG. 1, the veins 206 can be displayed in a manner distinguishing the veins 206 from the surrounding regions (e.g., tissue). For example, the veins 206 can be displayed as dark regions of the image 212 because more of the NIR light was absorbed by the veins 206 than by the surrounding tissue. Thus, the imaging system 200 can enable a medical practitioner to identify the location of a blood vessel 206 to facilitate placement of a needle therein. The imaging system 200 can be configured to provide real time vein imaging with no perceptible time delay. In FIG. 1, the imaging system 200 is shown twice. On a left portion of FIG. 1, the imaging system 200 is shown emitting light from the light source 202. On a right portion of FIG. 1, the imaging system 200 is shown receiving light from the target area 204 (e.g., reflected or scattered therefrom) onto the light sensor 208. Although shown separately in FIG. 1 for easy of illustration, in some embodiments light can be emitted from the light source 202 and received by the light sensor 208 simultaneously. In some embodiments, the light source 202 and the light sensor 208 can be disposed adjacent or near each other, e.g., on an imaging head 214 that includes both the light source 202 and the light sensor 208. Accordingly, in some embodiments, the light source 202 and the light sensor 208 can be coupled so that they are positionable together as a unit. In some embodiments, the light source 202 and light sensor 208 can be spaced apart from each other and can be independently positionable.

In some embodiments, the imaging system 200 can have sufficient accuracy and/or can have sufficient viewing depth into the patient's tissue to display infiltration or extravasation. Improved accuracy and viewing depth can also enable the imaging system to image veins 206 that are smaller in size and/or located deeper in the patient's tissue. To check patency of the vein, the medical practitioner can infuse a fluid (e.g., saline) into the vein and can image the vein using the imaging system. As used herein, the term “patency” is sometimes used to refer to whether or not a vein 206 is acceptable for infusion of a medical fluid. For example, if a vein 206 is open and has acceptable flow and is not ruptured, such that if a medical fluid is infused into the vein 206 the medical fluid will enter the patient's vascular system as intended, the vein 206 can be referenced as being patent or as having positive patency. However, if a vein 206 is compromised such that an attempt to infuse a medical fluid into the vein 206 will not result in the medical fluid entering the patient's vascular system as intended, the vein 206 can be referenced herein as lacking patency. As used herein a vein 206 can be compromised, such that it lacks patency, if the vein 206 is occluded or if the vein is ruptured or otherwise allows fluid to leak out of the vein 206 and into the surrounding tissue (e.g., resulting in infiltration or extravasation). Accordingly, in some embodiments, an assessment of the patency of a vein 206 can include determining whether infiltration or extravasation is present (e.g. in the body tissue surrounding the vein 206).

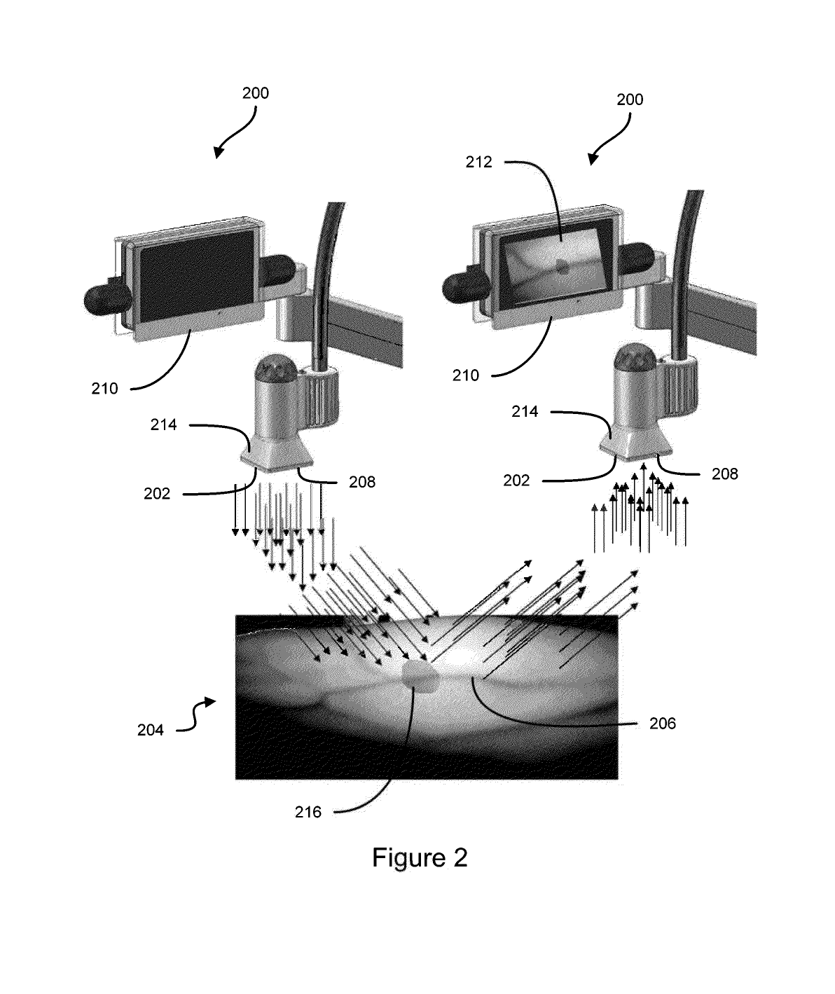

If the vein 206 is patent (e.g., as shown in FIG. 1), the image 212 will show the fluid (e.g., the infused fluid) contained within the vein 206 and/or show the fluid (e.g., saline) progress down the vein 206. If the vein 206 has been compromised, the fluid can leak out of the vein 206 and into the surrounding tissue. In some embodiments, blood can leak out of the vein 206 along with the infusion fluid, and the leaked blood (e.g., the hemoglobin therein) can absorb the NIR light so that the leakage 216 is visible in the image, for example as a dark area (e.g., as shown in FIG. 2). In some embodiments, the infused fluid can absorb the NIR light, so that infused fluid leaks out of the vein is visible as a dark areas in the image (e.g., as shown in FIG. 2). In some embodiments, an imaging enhancement agent can be infused (e.g., via an infusion site, which can include an intravenous (IV) line) to enhance the imaging of the vein 106 or of the infiltration or extravasation 216. For example, in some embodiments, the infused fluid can include a contrast agent or marker that increases the absorption of NIR light by the infused fluid. The imaging enhancement agent can be a biocompatible dye or a biocompatible near infrared fluorescent (NIRF) material. For example, the imaging enhancement agent can be NIRF dye molecules, NIRF quantum dots, NIRF single walled carbon nanotubes, NIRF rare earth metal compounds, etc. In some embodiments, the imaging enhancement agent can be Indocyanine Green. In some embodiments, the imaging enhancement agent can absorb NIR light (e.g., light having a wavelength between about 700 nm to about 1000 nm), and the imaging enhancement agent can fluoresce in the visible range or in the near infrared range, for example. If the imaging enhancement agent is configured to emit light in the visible range, the light emitted from the imaging enhancement agent can be observed without the use of a camera, although a camera may be used in some embodiments to capture an image of the infusion site. For example, due to the visible light output by the imaging enhancement agent in response to the NIR light from the light source, a user can observe the position of the imaging enhancement agent as it travels along the vein 106 in the area that is illuminated by the NIR light source. If infiltration or extravasation is present at the infusion site, the imaging enhancement agent can leak out of the vein and can be visible to the user when the infusion site is illuminated with NIR light. In some embodiments, a light sensor can be used to capture an image that includes the light emitted by the imaging enhancement agent. In some embodiments, the imaging enhancement agent can be configured to emit non-visible light and the camera can be sensitive to the wavelengths of light emitted by the imaging enhancement agent. A display can display the image to a user, e.g., to enable the user to make an assessment regarding the patency of the vein or regarding the presence or absence or extent of infiltration or extravasation. In some embodiments, the system can perform image processing on the image to automatically make an assessment of the patency of the vein or of the presence or absence of infiltration or extravasation. Various types of light sources can be used, such as LEDs, laser diodes, vertical-cavity surface-emitting lasers (VCSEL), halogen lights, incandescent lights, or combinations thereof. The light source can emit NIR light having a wavelength between about 700 nm and about 1000 nm, or of at least about 800 nm, at least about 830 nm, at least about 850 nm, at least about 870 nm, at least about 900 nm, or at least about 940 nm, although other ranges of wavelengths can be used. NIR light of longer wavelengths can penetrate deeper into the tissue of the patient because the light of longer wavelengths is less absorbed by the tissue, enabling imaging of deep infiltration or extravasation (e.g., due to leakage from the underside of a vein). However, the light sensor can be less sensitive to NIR light of longer wavelengths and/or the absorbing material (e.g., hemoglobin) can be less absorptive for NIR light of longer wavelengths, so that longer wavelength NIR light produces more degraded images. In some embodiments, the light source can emit NIR light between about 850 nm and 870 nm, which in some cases can provide sufficient accuracy and sufficient depth for imaging infiltration or extravasation. In some embodiments, short wave infrared (SWIR) light can be used, e.g., having wavelengths between about 1000 nm and 2,500 nm. In some embodiments, the light source can emit light between about 1000 nm and about 1050 nm, or of about 1030 nm.

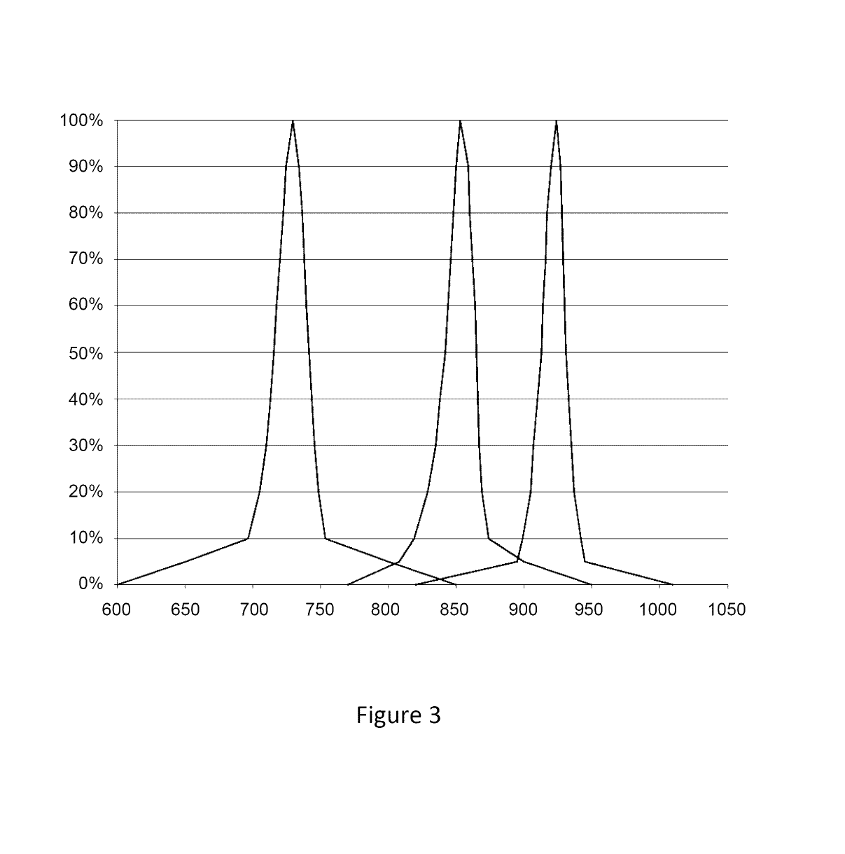

In some embodiments, the light source 202 can emit multiple wavelengths of light. For example, the light source can include three different types of light emitters (e.g., LEDs) that are configured to emit three different wavelengths of light. Although some embodiments are discussed as having three different light emitter types with three different wavelengths that produce three different image contributions, any number of light emitter types, wavelengths, and image contributions can be used (e.g., 2, 4, 5, 6, etc.). For example, 2, 3, or 4 types of light emitters (e.g., LED sets) can be used to emit light of different wavelengths ranging from about 700 nm to about 1000 nm, and in some embodiments, the light emitters can be pulsed or sequenced, as discussed herein. FIG. 3 shows a graph showing representative spectral outputs for three example types of light emitters (e.g., LEDs) having spectral peaks at about 730 nm, about 850 nm, and about 920 nm, respectively. Various spectral outputs can be used. For example, the light emitters can have nominal wavelengths of about 740 nm, about 850 nm, and about 950 nm respectively. In some embodiments, a first light emitter can emit light at about 700 nm to about 800 nm (e.g., about 750 nm to about 760 nm). A second light emitter can emit light at about 800 nm to about 900 nm (e.g., about 850 nm to about 870 nm). A third light emitter can emit light at about 900 nm to about 1100 nm (e.g., about 940 nm to about 950 nm). In some embodiments, the spectral output of the light emitters can have bell curve (e.g., Gaussian) shapes. In some embodiments, the spectral output curves for the different light emitters can overlap each other, as can be seen in FIG. 3. Light from the first light emitter can be used to produce a first image contribution of high quality but that reaches only a short distance into the tissue depth. Light from the second light source can be used to produce a second image contribution that has lower quality than the first image but reaches deeper into the tissue than the first image contribution. Light from the third light source can be used to produce a third image contribution that is able to reach deeper into the tissue than the first and second image contributions but has a lower quality than the first and second image contributions. In some embodiments some or all of the multiple light sources can emit light with a wavelength between about 1000 nm and about 2500 nm.

In some embodiments, all three light emitters can be turned on at the same time so that the light from all three light emitters illuminates the target area simultaneously. Light of all three wavelengths can be reflected or scattered by the target area to the light sensor 208 to produce a single composite image that is a combination of the three image contributions. In some embodiments, a single broadband NIR light source can be used instead of multiple distinct light source types.

In some embodiments, the light emitters can be pulsed in sequence with the light sensor (e.g., synchronized with a shutter of the camera), so that the light emitters are turned off when the light sensor is not generating an image and so that the light emitters are turned on when the light sensor is generating an image. In some cases, the pulsing of the light emitters can be synchronized with the shutter of the camera so that the light emitters are turned on when the shutter is open and turned off when the shutter is closed. Turning the light emitters off when not needed can reduce power usage and heat buildup. In some embodiments, a light source 202 that includes only a single light emitter, or light emitters of all substantially the same wavelength, or of different wavelengths, can be pulsed at a rate that corresponds to an imaging rate of the light sensor 208.

In some embodiments, the light emitters can be pulsed sequentially. For example, at a first time, the first light emitter can be turned on while the second and third light emitters are turned off, and the light sensor can generate a first image at the first time using the light from the first light emitter. At a second time, the second light emitter can be turned on while the first and third light emitters are turned off, and the light sensor can generate a second image at the second time using the light from the second light emitter. At a third time, the third light emitter can be turned on while the first and second light emitters are turned off, and the light sensor can generate a third image at the third time using the light from the third light emitter. As mentioned above, additional images can be generated by additional light emitters of different wavelengths, depending on the number different wavelengths utilized. The different images can be displayed on the display device in rapid succession (e.g., interlaced) so that the images combine to form a composite image of all three images to the human eye. Similarly, the different images can be stored in memory and then combined by the imaging system to form a composite image, which may be displayed on the display device to the user. Optionally, a control may be provided enabling the user to instruct the imaging system to display each image individually and/or to display a composite image including images selected by the user.

Pulsing the light emitters sequentially can allow for more light of each wavelength to be used. For example, if all three light emitters are turned on together, the amount of light emitted by each light emitter may need to be limited or reduced to avoid overpowering the light sensor. However, if the light emitters are pulsed sequentially, the more light of each wavelength can be used since the light is not combined with the other wavelengths of light from the other light emitters. By illuminating the target area with more light of each of the three light emitters, the quality and/or imaging depth of the produced image can be improved. In some sequentially pulsing embodiments, the light sensor can be configured to capture images at a faster rate (e.g., 60 hz or 90 hz) than would be needed in embodiments in which the light emitters are turned on together, since the different image portions are captured separately. In some embodiments, the light sensor 208 can include multiple light sensor portions (e.g., as subpixels of the light sensor 208) configured to synchronize with the multiple light emitters that are pulsed in sequence. In some embodiments, different light sensors can be used for the different wavelengths of light and can be configured to synchronize with the pulsing of the multiple light emitters.

The composite image 212 that includes the three image portions can provide the benefits of all three image portions to the user simultaneously, without requiring that the user toggle between the different wavelengths of light. When the user wants to observe a feature that is relatively deep in the tissue, the user can focus on the third image portion of the composite image, which is produced using the longer wavelength NIR light. When the user wants to observe high quality detail of a feature that is relatively shallow in the tissue, the user can focus on the first image portion of the composite image, which is produced using the shorter wavelength NIR light. Although the presence of the third image portion can degrade the quality of the first image portion to some degree, it is expected that the human mind is able to focus on the desired portions of the image while deemphasizing the other portions of the image. Various embodiments disclosed herein can utilize a light source 202 that is configured to pulse, as discussed herein, and can include multiple light emitters for producing images with different wavelengths of light, even where not specifically mentioned in connection with the specific embodiments.

In some configurations, the light source 202 can emit light having irradiance of at least about 5 mW/cm² and/or no more than about 7 mW/cm², at a distance of about 100 mm from the light source, at a given time, although irradiance outside these ranges can also be used (e.g., depending on the sensitivity and configuration of the light sensor). Higher power output can increase the quality of the produced image and/or enable the system to image deeper into the tissue of the patient. However, if too much light is used, the light sensor can be oversaturated. The amount of light that the light source 202 outputs can depend on the distance between the light source 202 and the target area. For example, less light intensity can be used when the light source is disposed closer to the target area, and more light intensity can be used when the light source is disposed further from the target area. In some cases the system 200, and various other systems disclosed herein, can be configured to operate at a distance of at least about 100 mm, at least about 150 mm, at least about 175 mm, at least about 190 mm, at least about 200 mm, less than or equal to about 300 mm, less than or equal to about 250 mm, less than or equal to about 225 mm, less than or equal to about 210 mm, or less than or equal to about 200 mm.

The imaging system can include an optical filter to block undesired light. For example, the filter can be configured to transmit light of the wavelength range emitted by the light source while attenuating light outside the wavelength range emitted by the light source 202. The filter can be a narrow bandpass filter that transmits only a narrow range of desired wavelengths, or the filter can be a longpass filter that attenuates light (e.g., visible light) that has a lower wavelength than the NIR light emitted by the light source 202. The filter can be an absorptive filter, an interference filter, a multi-layer thin film filter, or any other suitable filter type. The optical filter can be incorporated into the optical system in various manners. For example, the optical filter can be disposed in front of a camera lens or behind the camera lens (e.g., over the light sensor). In some embodiments, the optical filter can be applied directly onto one or more surfaces of the camera lens (e.g., as a thin film interference stack deposited onto the lens).

In some embodiments, multiple optical filters can be used. For example, if the light source 202 includes multiple light emitter types, multiple optical filters can be used that are configured to transmit wavelengths of light associated with the corresponding light emitter. In some embodiments, a first optical filter can transmit the wavelengths of light emitted by the first light emitter and can attenuate other wavelengths of light, a second optical filter can transmit the wavelengths of light emitted by the second light emitter and can attenuate other wavelengths of light, and a third optical filter can transmit the wavelengths of light emitted by the third light emitter and can attenuate other wavelengths of light. The optical filters can be disposed over different light sensor portions associated with the different light emitter types, or a single light sensor portion can be used and the optical filters can be actuated (e.g., using a filter wheel) or switched in synchronization with the light emitters so that the first optical filter is disposed to filter light for the light sensor at the first time, the second optical filter is disposed to filter light for the light sensor at the second time, and the third optical filter is disposed to filter light for the light sensor at the third time.

Blocking unused light from reaching the light sensor can improve the quality of the image and can allow the light source to emit more light without oversaturating the light sensor. In some embodiments, the imaging system can include a polarizing filter, which can be configured to reduce glare (e.g., reflected from the surface of the patient's skin), thereby further improving the image quality and allowing for the light source to emit more light. For example, the polarizer can be oriented to block s-polarized light from the surface of the patient's arm (or other body portion) in its expected position, e.g., horizontally disposed. In some embodiments, glare and/or unused wavelengths of light can be attenuated, thereby reducing the amount of glare and/or unused wavelengths of light that reaches the light sensor. This can improve the quality of the produced image. Reducing the amount of glare and/or unused light that reaches the light sensor can also allow the light source to emit more light without oversaturating the light sensor, further improving the quality of the image and/or allowing the image to penetrate deeper into the tissue of the patient. In some embodiments, the light source can emit light having an irradiance of at least about 10 mW/cm² and/or no more than about 20 mW/cm², at a distance of about 100 mm from the light source, although irradiance outside these ranges can also be used (e.g., depending on the sensitivity and configuration of the light sensor). Various embodiments disclosed herein can have an operating distance of between about 150 mm and about 250 mm.

In some embodiments, one or more optical elements (e.g., lenses) can adjust the light output from the light emitters, for example, to increase the amount of light emitted from the light source that reaches the target area. The one or more lenses can be a positive or converging lens that at least partially focuses the light from the light source onto the target area on the patient. For example, the one or more lenses can decrease divergence of the light or increase convergence of the light. In some embodiments, the camera (or any other part of circuitry of the imaging system) can include electrostatic shielding to reduce noise. In some embodiments, the camera and/or other components of the imaging system can include all-digital circuitry, which can produce images with less noise than analog circuits. The digital images may be processed by a processor to provide, for example, image processing. The processer can perform pre-processing operations and/or post-processing operations on the image. In some embodiments, the system does not include an analog to digital (AD) converter for processing the image data, e.g., since all-digital circuitry can be used. In some embodiments, a digital display 210 can be used to display the image 212, and a digital-format cable can be used to provide a digital communication link between the light sensor 208 and the display 210.

In some embodiments, the light sensor 208 can be sufficiently sensitive to light of the wavelengths emitted by the light source 202 to image the veins 206 and/or infiltration or extravasation 216, as discussed herein. For example, the light sensor 208 can be substantially sensitive to light having a wavelength of at least about 800 nm, at least about 830 nm, at least about 850 nm, at least about 870 nm, at least about 900 nm, or at least about 940 nm. In some embodiments, the light sensor 208 can be an indium gallium arsenide (InGaAs) light sensor, a charge-coupled device (CCD) sensor, or a complementary metal-oxide semiconductor (CMOS) sensor.

In some embodiments, the imaging system can perform image processing (e.g., digital image processing) to reduce noise or otherwise improve the displayed image 212. The image processing can include noise reduction to improve the quality of the image 212. The image processing can include edge sharpening, which can emphasize the edges of the veins 206 and/or the edges of the fluid 216 leaked from the vein in the image 212. The image processing can include contrast enhancement that can darken the veins 206 or leaked fluid 216 and/or can lighten the tissue surrounding the veins in the image 212. In some embodiments, the image processing can include gamma correction. The image processing can also modify the image 212 based on lookup tables. In some embodiments, the grayscale image 212 can be colorized. For example, a first color (e.g., blue) can be applied to portions of the image 212 (e.g., pixels) that are above a threshold brightness level, indicating that the portion of the image 212 is associated with tissue surrounding the veins 206. A second color (e.g., red) an be applied to portions of the image 212 (e.g., pixels) that are below a threshold brightness level, indicating that the portion of the image 212 is associated with a vein 206, and/or with leaked fluid 216 resulting from infiltration or extravasation. A lookup table (LUT) can be used for the colorization process. The LUT can include image information (e.g., color information, brightness information) for various values (e.g., brightness values) in the original image 212. Thus, the pixels of the original image 212 can be mapped to new values based on the LUT to produce a processed (e.g., colorized) version of the image 212.

In some embodiments, various settings can be adjusted depending on the environment, patient health, site availability, preferences of the medical practitioner, etc., such as the light source 202 power, the camera settings (e.g., shutter speed), the angle and height of the light source 202 and/or camera 208.

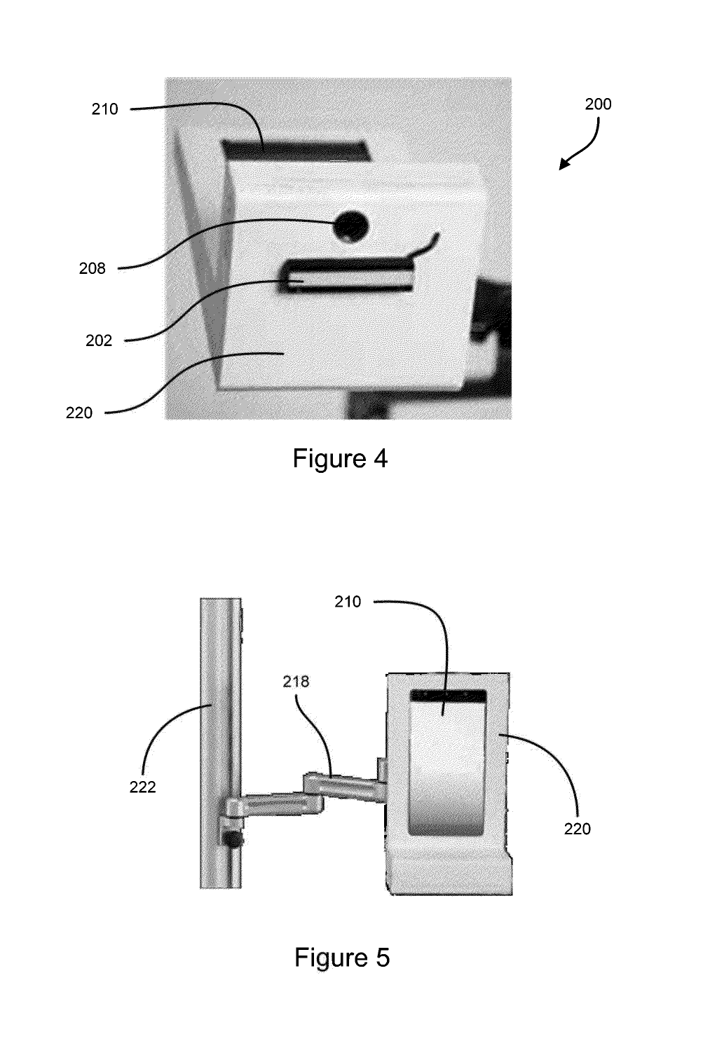

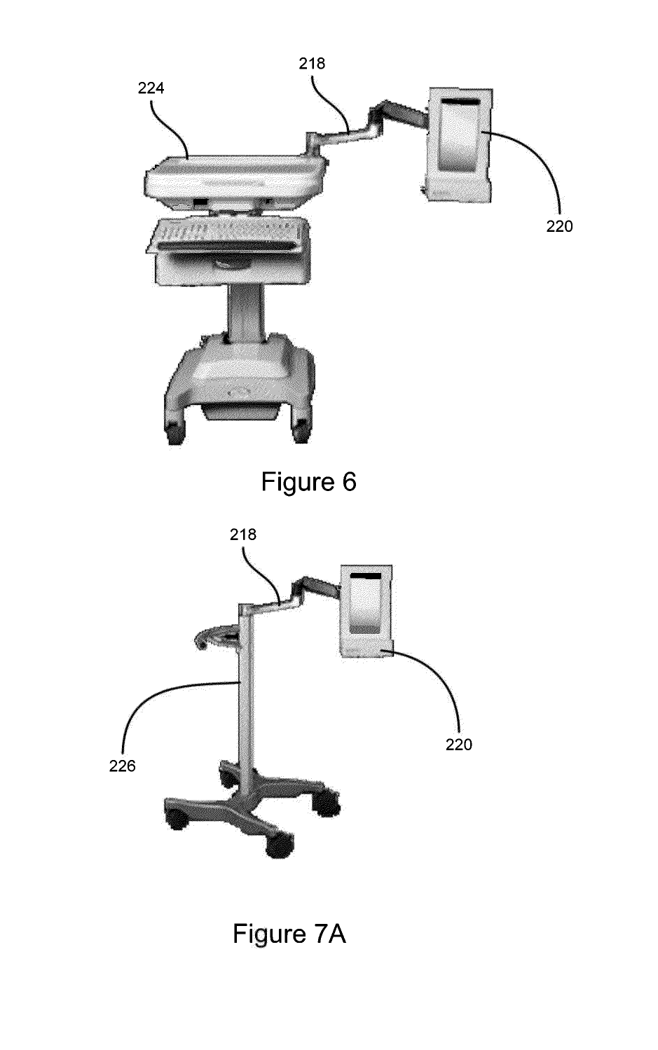

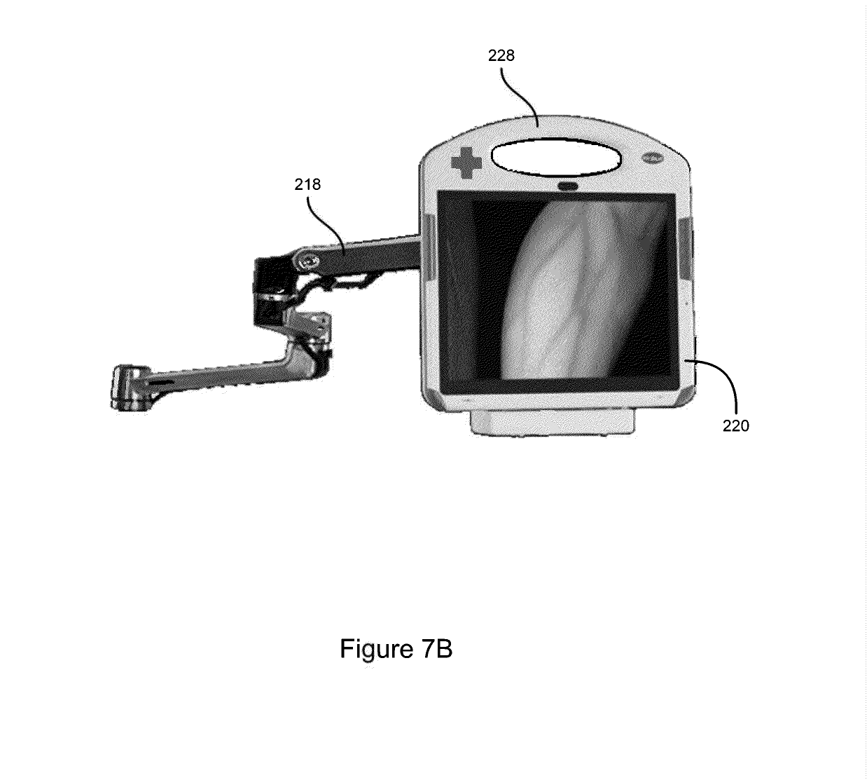

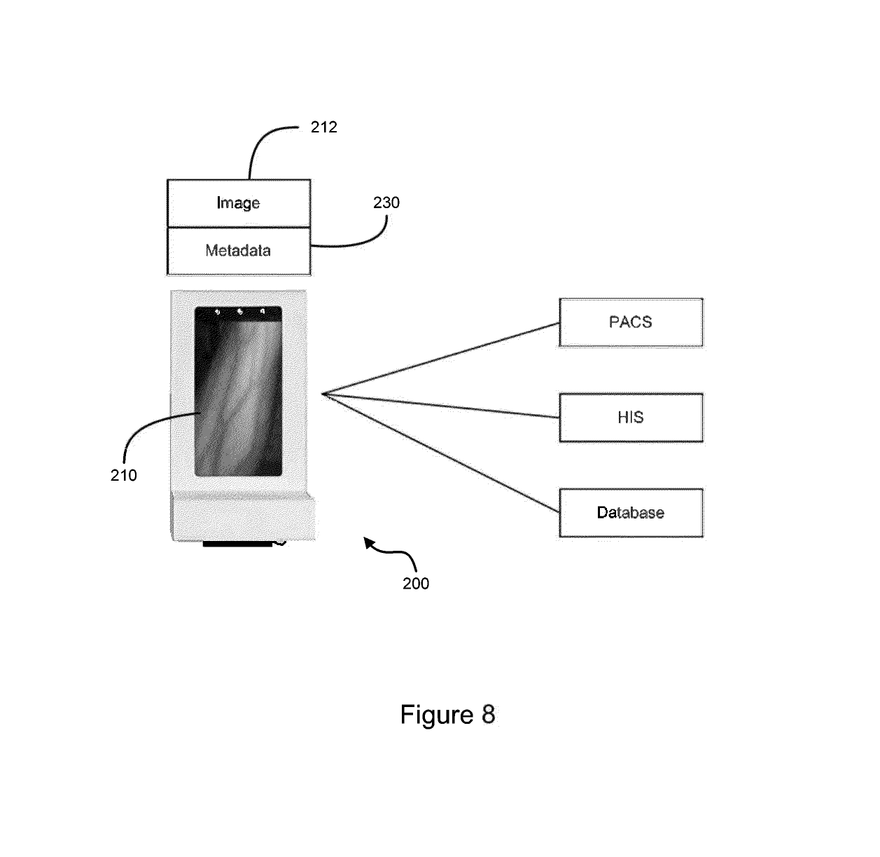

As shown in FIG. 4, in some embodiments, the light source 202, the light sensor 208 (e.g., camera), and display device 210 can be incorporated into a single integrated device 220 (e.g., sharing a single housing), or the lights and/or camera can be remote from the emitter and/or receiver and can be connected by one or more light guides (e.g., fiber optic bundles). The light emitters can be placed next to or around the camera 208. The integrated device can be mountable onto an articulating arm 218, which can be slidably coupled to a vertical support member 222, where the height of the articulated arm 218 (and the integrated device 220) may be vertically adjusted, as shown in FIG. 5, to enable the device to be positioned in a wide variety of positions depending on the patient's orientation, the medical practitioner's position, the portion of the patient's body being imaged, etc. The device can be mounted onto a point of care cart 224 (e.g., FIG. 6), onto a clinic utility cart 226 (e.g., FIG. 7A), or in various other configurations. In some embodiments, the device can be a handheld device (e.g., a tablet or other mobile device). For example, FIG. 7B shows an example embodiment of a mobile device 220 that incorporates the imaging system 200. The mobile device 220 can be a tablet, in some embodiments. The device 220 can have a handle 228. In some embodiments, the device 220 and the support member (e.g., articulated arm 218) can be coupled together by a quick release mechanism that allows a user to quickly release the device 220 from the support member (e.g., articulated arm 218). In some embodiments, the device can be wearable, for example, as a head mounted display, mounted onto a forearm of a user, as a necklace or pendant, or in various other configurations as discussed herein.

Medical practitioners often check the patency of a vein (e.g., periodically or in connection with IV treatment such as infusion of medication). Because it can be difficult to accurately determining whether a vein has been compromised, especially for small amounts of infiltration or extravasation, errors in assessing patency sometimes occur, which can cause harm to the patient. Since the imaging system 200 can accurately image the patient's vasculature, including the presence or absence of infiltration or extravasation, the imaging system 200 can provide a more objective, more definitive, more quantifiable (e.g., due to ability to measure size of leakage), and more documentable (e.g., due to ability to store image of leakage) basis for the medical practitioner to use to determine vein patency.

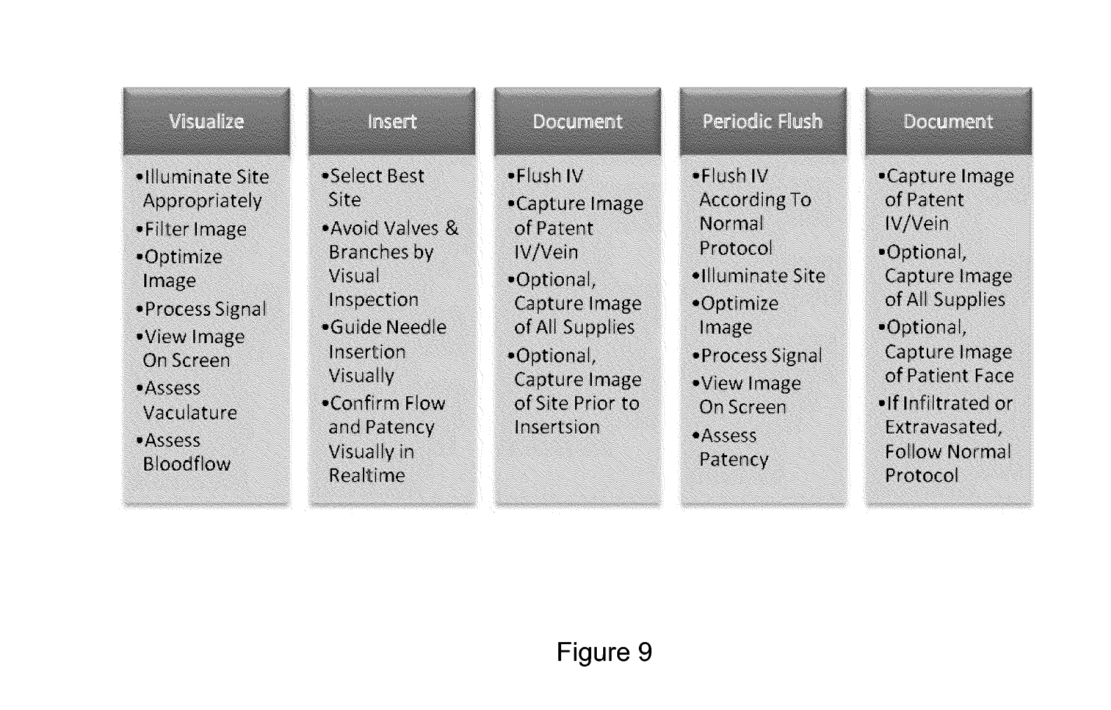

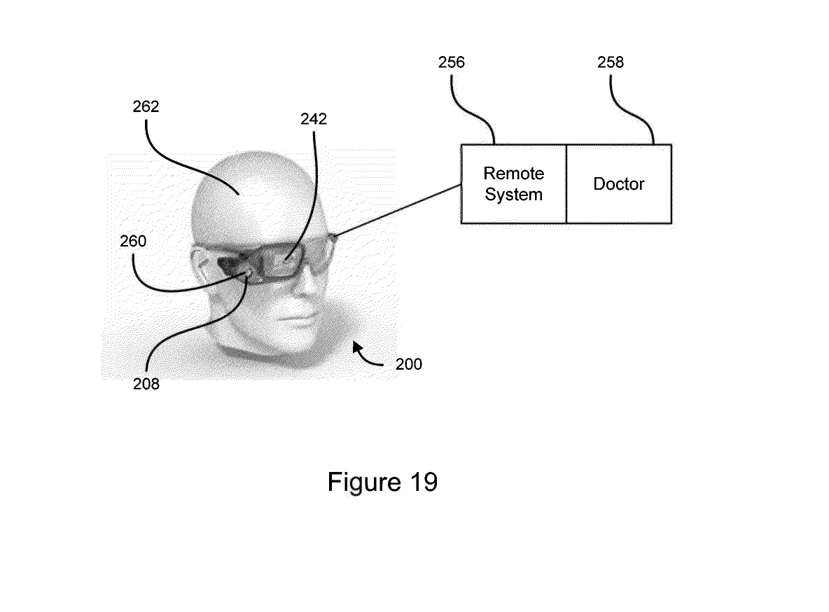

With reference to FIG. 8, in some embodiments, the imaging system 200 can be used to document the status of a patient's vein 206 and/or an IV connection. The imaging system 200 can store, in a computer-readable memory, an image of the patient's vasculature that shows the presence or absence of infiltration or extravasation. For example, a medical practitioner can check the patency of a vein and/or IV connection by infusing a fluid (e.g., saline) and imaging the area around the infusion site with the imaging system 200. If the image 212 displayed or provided by the imaging system 200 does not show infiltration or extravasation, the medical practitioner can make the determination that the vein and/or IV connection is patent. The medical practitioner can provide a command to the imaging system 200 (e.g., by pressing a button) to store a copy of the image showing the absence of infiltration or extravasation. In some embodiments, the medical practitioner can provide input to the system and can indicate whether the vein and/or IV connection was determined to be patent or compromised. The system can prompt the user to provide an indication of whether the vein and/or IV connection was determined to be patent or compromised, such as using the display 210. The user can provide input and commands to the system via a touchpad keypad (e.g., as shown in FIG. 16), an external keyboard (e.g., as shown in FIG. 6), a touchscreen display device, voice commands, or otherwise. If the medical practitioner determines, based on the displayed image, that the vein and/or IV connection has been compromised, the medical practitioner can provide a command (e.g., by pressing a button) to store a copy of the image showing the presence of infiltration or extravasation.

In response to the command, or another command, the system can associate information (e.g., as metadata 230) to the image 212. The information associated with the image 212 (e.g., as metadata 230) can include an identifier of the patient, which can be input, by way of example, to the system by using a bar code scanner or the device's camera to read a bar code (e.g., a 1D or 2D barcode) or other label associated with the patient (e.g., on a wrist band worn by the patient), via an RFID scanner reading the information from an RFID tag worn by the patient, via a fingerprint reader, or by a user manually entering the information using an input device, such as those discussed elsewhere herein. Patient information can be populated from the electronic medical records (EMR), or from the information on the wrist band or other label, or from manual entry. The patient identifier can be a picture of the patient's face, in some embodiments. The information associated with the image 212 (e.g., as metadata 230) can include an identifier of the medical practitioner who performs the patency check, which can be input by scanning a bar code or other label associated with the medical practitioner, or via a fingerprint reader, or any other suitable device. In some embodiments, the medical practitioner can input information (e.g., metadata 230) (such as a patient name or other identifier, gender, age, health condition, operator, name, or other information) using a touchpad keypad, external keyboard, or touchscreen, etc. The information associated with the image 212 (e.g., as metadata 230) can include time information, such as the date and time that the image was recorded. The information (e.g., metadata 230) and the image 212 can be incorporated into a single file, or the information (e.g., metadata 230) can be stored separately from the image 212 and can be linked to the associated image. The information (e.g., metadata 230) can be associated directly with the image 212 by use of a header, e.g., having multiple fields. In some embodiments, the image 212 and can be stored in a patient file or folder (e.g., in an electronic patient file or in a physical file or folder associated with the patient). Storage of an image 212 in a patient file or folder can associate the image 212 with the patient. Accordingly, the folder or file in which an image 212 is stored can serve as the patient identifier information associated with the image 212. The information associated with the image 212 (e.g., metadata 230) can include an indication of whether the vein and/or IV connection was determined to be patent or compromised. In some embodiments, a picture of the medical materials used can be stored to document the procedure performed by the medical practitioner. In some embodiments, the information associated with the image 212 (e.g., metadata 230) can allow the images 212 to be indexed or searched (e.g., by patient identifier, by medical practitioner identifier, by time information, etc.).

Various file formats and storage systems can be used. In some embodiments, the images 212 and/or the associated information (e.g., metadata 230) can be encrypted, transferred, and/or stored in accordance with digital imaging and communications in medicine (DICOM) standards. The images 212 and/or the associated information (e.g., metadata 230) can be stored in a picture archiving and communication system (PACS), which can be incorporated into a hospital information system (HIS), which can include electronic medical records (EMR), in some embodiments. Thus, the images and/or the metadata may be stored locally to the imaging system and/or remotely on another system. The imaging system 200 can include a communication system that is configured to send information to and/or receive information from various other components and systems (as discussed herein) via a wireless communication link, one or more cables, or in any other suitable manner.

The imaging system 200 can reduce patency check errors by enabling the medical practitioner to view the patient's vasculature and the presence or absence of infiltration or extravasation. The imaging system 200 can also generate and provide documentation (e.g., autonomous documentation, since, for example, the system independently posts the date and time stamp) that the medical practitioner performed the patency check as well as information that confirms that the patency check was accurate. Thus, if the need arises to determine whether a patency check was performed (e.g., during a medical malpractice lawsuit or other claims of medical error), the image 212 and associated information (e.g., metadata 230) can be consulted to determine whether the patency check was performed and to confirm that the patency check was accurate. By documenting that the patency check was properly performed, the system can reduce risk of medical malpractice liability associated with treating the patient. The documentation can also be useful to consult when a medical practitioner makes decisions regarding patient treatment (e.g., whether to replace an IV line). The medical practitioner can document the patency of the vein and/or IV connection as part of the procedure for initially establishing the IV connection, when periodically flushing the IV connection and/or vein with fluid (e.g., saline) according to standard IV protocol, and/or or as part of an IV treatment procedure such as infusion fluid into the IV connection and/or vein or drawing bodily fluid therefrom. The system may be configured to automatically generate a report requested by a user, including a patient name, unique identifier, date/time of the examination/procedure, patient demographics (age, gender, etc.), reported health issue, images, image date, operator name, other metadata, etc. The report may be displayed, printed, and/or electronically transmitted.

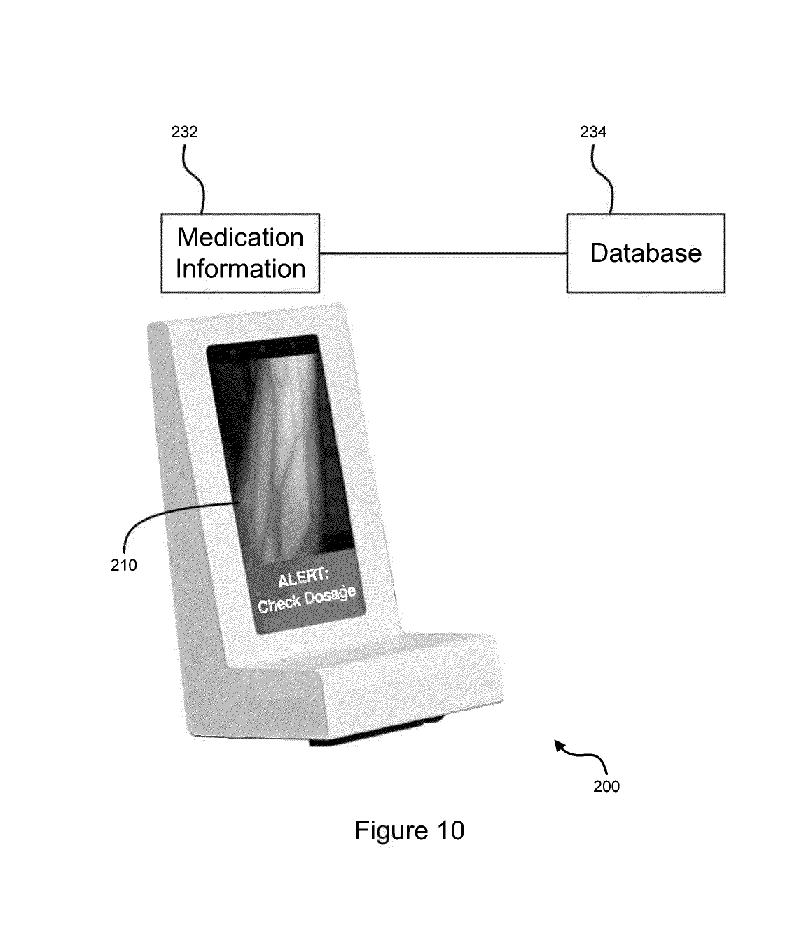

FIG. 9 is a flowchart showing an example method of operating the system. One or more medical practitioners can perform the various steps set forth in FIG. 9 and/or various steps may be performed by the system. Various steps described in FIG. 9 can be omitted or rearranged to form different combinations and subcombinations of the method steps shown. In some embodiments, the imaging system 200 can be used to visualize the patient's vasculature. The imaging system 200 can illuminate a site on a patient (e.g., using the light source 202). The light can be received by a light sensor (e.g., after being reflected or scattered by the site on the patient). In some embodiments, one or more optical filters can filter light received by a light sensor 208, which can improve the resulting image 212. The image 212 can be optimized, e.g., by image processing (e.g., digital pre-processing and/or digital post-processing) performed by a processor. The image 212 can be displayed on a screen, so that the image 212 can be viewed by a medial practitioner. The medical practitioner can view the image 212 and assess the vasculature of the patient at the site being imaged. In some embodiments, the medical practitioner can assess blood flow in one or more veins based on the image 212 presented on the screen.

In some embodiments, the image system can be used to facilitate insertion of an intravenous (IV) line. For example, a medical practitioner can select a location for the IV line, e.g., based at least in part on the displayed image of the patient's vasculature. The presented image 212 can enable a medical practitioner to avoid branches and valves and other problematic areas when selecting a location for the IV line. The image 212 can also be used during insertion of the IV line to facilitate positioning of the needle into the selected vein. In some cases, once a needle or IV line has been inserted, the medical practitioner can use the imaging system to confirm patency of the vein, IV line, and/or infusion site. For example the medical practitioner can infuse a fluid into the IV line and can visually confirm flow of the infused fluid in the vein and/or the absence of infiltration and extravasation. In some embodiments, the user can infuse a fluid (e.g., saline) that is configured to scatter or reflect less light than the blood in the vein. Accordingly, the fluid (e.g., saline) can be visualized on the image 212 as a bright area (as compared to the dark areas that correspond to blood in the veins). If the vein is patent and has good flow, the bright area associated with the fluid (e.g., saline) in the image will move along the vein as the flow of blood transports the fluid (e.g., saline). Accordingly, the imaging device 200 can be used for assessing the flow in a vein (e.g., to confirm that a vein is not occluded). The imaging system 200 can also be used to image the infusion site to confirm that no infiltration or extravasation is present, as discussed herein.

In some embodiments, an imaging enhancement agent can be infused into the infusion site and can be used for assessing patency, as discussed herein. For example, the imaging system 200 can be used to illuminate the infusion site with NIR light, and the infused imaging enhancement agent (which can be an NIR fluorescent material) can be configured to absorb the NIR light from the imaging system 200 and to emit light of a wavelength different than the wavelength output by the imaging system 200. In some embodiments, the light emitted by the imaging enhancement agent can be visible light, which can enable a user to view the location of the imaging enhancement agent directly (e.g., with a light sensor 208 and display screen 210). For example, the user can observe that the location that emits visible light moves generally linearly along the body portion of the patient away from the infusion site, which can be an indication that the vein is not occluded and has acceptable flow. If the user observes that the location that emits visible light (e.g., by fluorescence) does not travel away from the infusion site, or that the area that emits the visible light covers an area that indicates that the fluid has leaked out of the vein, that can be an indication that the vein is occluded, ruptured, or otherwise compromised. In some systems that utilize an imaging contrast agent, a light sensor 208 and display 210 can be used to assess the vein. For example, an imaging contrast agent can be used to is fluorescent and emits non-visible light, which can be used by the imaging system 200 to generate the image 212.

In some embodiments, the imaging system 200 can be used to document the infusion site. As discussed herein, a medical practitioner can flush the IV line, e.g., by infusing a fluid into the infusion site, and the imaging system 200 can be used to visualize the presence, absence, or extent of infiltration or extravasation. The imaging system 200 can capture one or more images 212 that show the presence, absence, or extent of infiltration or extravasation. The one or more images 212 can be stored (e.g., in a patient treatment archive), and the one or more images 212 can be associated with information such as a patient identifier, time information, and a medical practitioner identifier, as discussed herein. In some embodiments, the imaging system 200 can be used to capture and store an image of the medical supplies used for inserting the IV line. The imaging system 200 can also be used to capture and store an image of the site on the patient before the IV line is inserted. These images can also be associated with information such as patient identifiers, medical practitioner identifiers, and time information, etc. Images associated with confirming blood flow can also be captured and stored, and can be associated with information such as a patient identifier, medical practitioner identifier, time information, etc., which can later allow the images to be indexed or searched. For example, to show flow of saline or of an imaging contrast agent that is infused into the IV line, multiple images can be saved showing the movement of the saline or imaging contrast agent along the vein. In some cases, video images can be captured and stored.

In some embodiments, the imaging system 200 can be used for periodic checks of the IV line. The IV line can be flushed, and the imaging system 200 can be used to illuminate the site (e.g., with NIR light from the light source 202). An image of the site can be obtain, optimized, and processed as described herein, and the image 212 an be presented on the display screen 210. The medical practitioner can view the image 212 and make an assessment of the patency of the vein based at least in part on the image 212. For example, the image 212 can be configured to show the presence, absence, or extent of infiltration or extravasation at the site. The image 212 can also be used to confirm blood flow and vein patency, as discussed herein.

In some embodiments, the imaging system 200 can be used to capture one or more images that show the presence, absence, or extent of infiltration or extravasation, and/or that show whether or not the vein has acceptable blood flow, as discussed herein. Information, e.g., patient identifiers, time information, medical practitioner identifiers, etc. can be associated with the one or more images. An image of the medical supplies used for the patency check can be captured and stored, and be associated with the information such as patient identifier, time information, and medical practitioner information. An image of the face of the patient can be captured and stored and can be associated with the information or with the other captured images as well.

If the assessment of the vein results in a determination that the vein is occluded, ruptured, or otherwise compromised, the medical practitioner can proceed with normal protocol (e.g., to replace the IV line).

The system can include a controller that can include one or more computer processors, which can be incorporated into the imaging system 200 (e.g., in the same housing as the light source 202, light sensor 208, and/or the display device 210). The one or more computer processors can be located remotely from one or more components of the imaging system, and the imaging system can include a communication link (e.g., via a cable or wireless connection, or combinations thereof) to the one or more computer processors. The one or more computer processors can be specially configured to perform the operations discussed herein. In some embodiments, the one or more computer processors can be in communication with computer-readable memory (e.g., a non-transitory computer readable medium) that includes computer-executable code (e.g., program modules) configured to cause the one or more computer processors to implement the operations discussed herein. Various embodiments that are discussed herein as being implemented with software can also be implemented using firmware or hardware components (e.g., integrated circuits), and vice versa. In some embodiments, multiple processors or computing devices can be used, such as for parallel processing.

In some embodiments, the system can be configured to verify medication information. Many medications are delivered intravenously. When a medical practitioner prepares to infuse a medication via an IV connection, the medication practitioner can check the patency of the vein and/or IV connection using the imaging system as discussed herein. Accordingly, the medical practitioner can use the imaging system at the patient's location and at a time just before administering the medication. By also using the system to verify medication information while the medical practitioner is at the patient's location and just before administering the medication, the risk of error can be decreased. For example, if a medication verification system is located in the hall or nurse station in a hospital, the inconvenience of using the medication verification system can result in the medical practitioners skipping the medication verification process. Also, even if a medical practitioner uses a remote medication verification system to confirm that the medication to be administered is correct, errors can occur between the remote medication verification system and the patient (e.g., by walking into the wrong patient's room). Thus, by incorporating a medical verification system into the imaging system that the medical practitioner uses at the patient's location and just before administering the medication or as part of the medication administration process itself, the likelihood of errors can be reduced.

The system 200 can be configured to receive information 232 about the medication being administered, such as the medication type, concentration, and volume. In some embodiments, the medication can be provided in a container (e.g., a syringe) that includes a bar code or other label that can be used to input the medication information into the system 200 (e.g., by a reading performed by a bar code scanner or by the system's camera). For example, the medication can be prepared by a hospital pharmacy, and a bar code or other label can be attached to the medication container to identify the medication as discussed above. In some embodiments, the medication does not include a barcode, but can have a label with a written description of the medication, and the written description can be photographed to document the medication administered to the patient. The system 200 can also be configured to receive a patient identifier (e.g., which can input as part of, or in like manner to, the patency check process discussed above). The system 200 can also be configured to receive an identifier of the medical practitioner (e.g., which can be input as part of, or in like manner to, the patency check process discussed above).

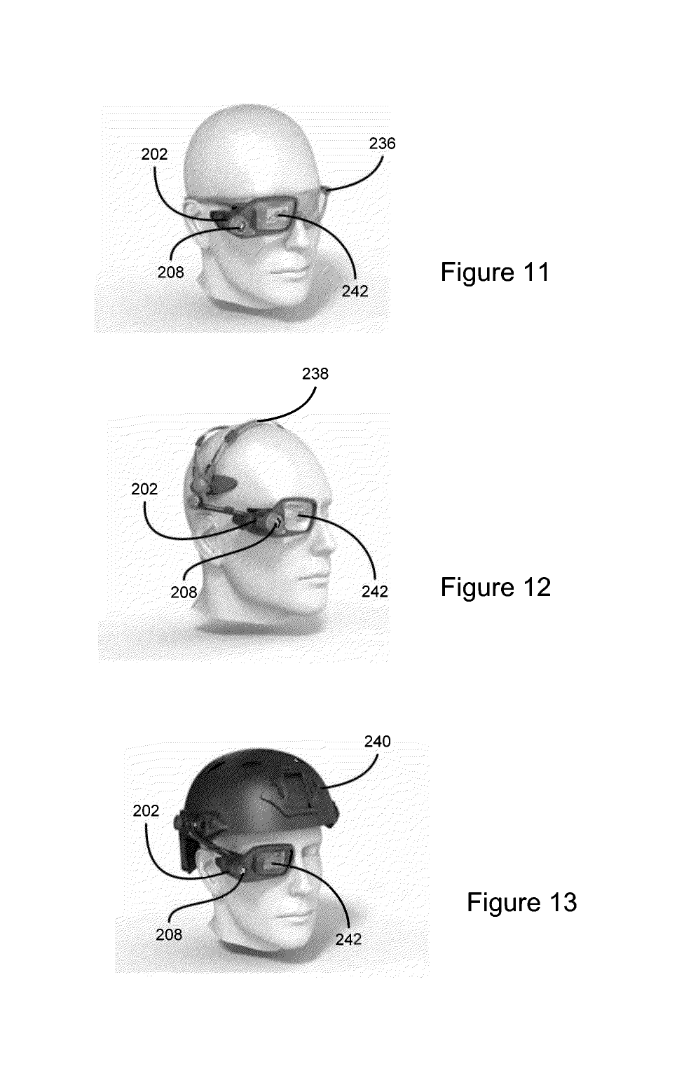

The system 200 can access one or more local or remote databases 234 of information and can determine whether to issue a warning based at least in part on the accessed information. For example, the database 234 of information can have information regarding expected dosage amounts, and the system 200 can issue a warning if the medication is for a dosage that is outside the usual dosage amount. For example, if the controller receives an indication that the medical practitioner plans to infuse a 50 mL of a particular drug (e.g., by scanning a bar code on the syringe containing the drug or by the user manually entering the information), the system can access information about the particular drug in the database to determine that a usual dosage for the particular drug ranges from 1 to 10 mL. Since 50 mL is outside the expected range, the system 200 can display a warning to the medical practitioner (e.g., as shown in FIG. 10). In some embodiments, the database 234 can be incorporated as part of the Hospital Information System (HIS), or the database 234 can be a separate database (e.g., a third party database). In some implementations, the system 200 can determine whether to issue a warning based on patient information, such as age, condition, prior medication, etc. Thus, the system 200 can be configured to recognize the scenario in which a drug has already been administered to a patient (to prevent duplicate administration of the drug). The system can recognize when a particular drug or dosage is not appropriate for the patient (e.g., an adult dosage for administration to a child, or a pregnancy medication to a cardiac patient). In some embodiments, the system 200 can access a prescription for the patient in the HIS to determine the proper administration of medication and can issue a warning if the medication that is about to be administered does not match the prescription.

In some embodiments, checking the medication information 232 to determine whether to issue a warning can be performed in response to the user providing a command to the system (e.g., the command to store an image of the patent vein). Thus, during use, the medical practitioner can check the vein for patency using the imaging system 200. Once patency of the vein is confirmed by the user, the user can provide a command to the system to store the image (e.g., by pushing a button). The command can also cause the system to check for potential warnings based on the medication information. If no warnings apply, the system can instruct the user to administer the medication. If a warning is applicable, the system can display the warning information to the user (see FIG. 10) and/or can transmit the warning to another destination (e.g., via an SMS, MMS, or email message to a supervisor phone or pager, and/or to a database). By using the same command to store the patency check image and to initiate the check for warnings on the medication, the system can cause that the medication be checked just prior to administration of the medication.

The system 200 can be configured to document the administration of the medication to the patient. The information 232 about the medication (e.g., medication type, volume, and concentration) can be stored in a database 234 along additional information, such as the patient identifier, the identifier of the medical practitioner, and the date and time. Thus, if a need later arises to review what medication was administered, the saved information can be consulted. In some embodiments, the system can be configured to save a picture of the patient and/or a picture of the medication about to be administered to the patient.

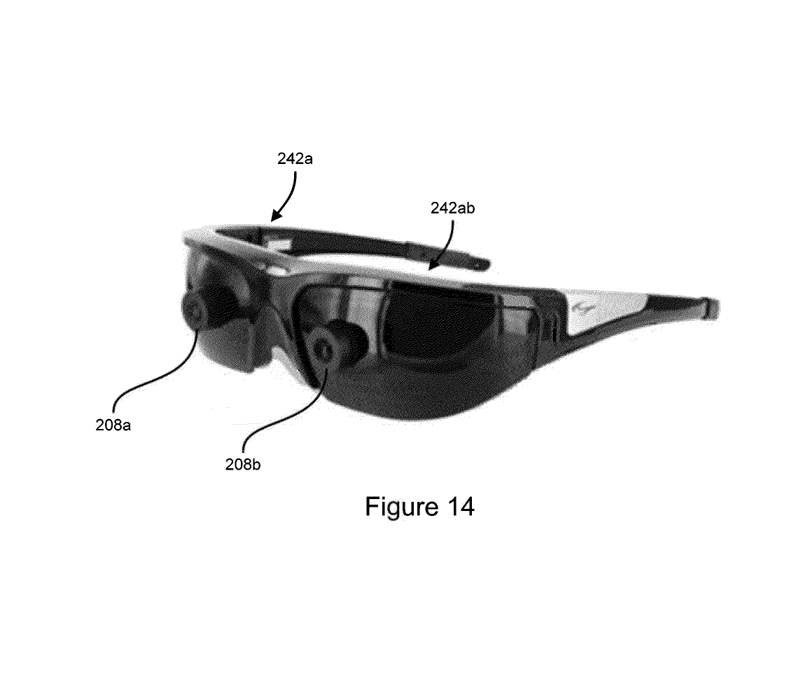

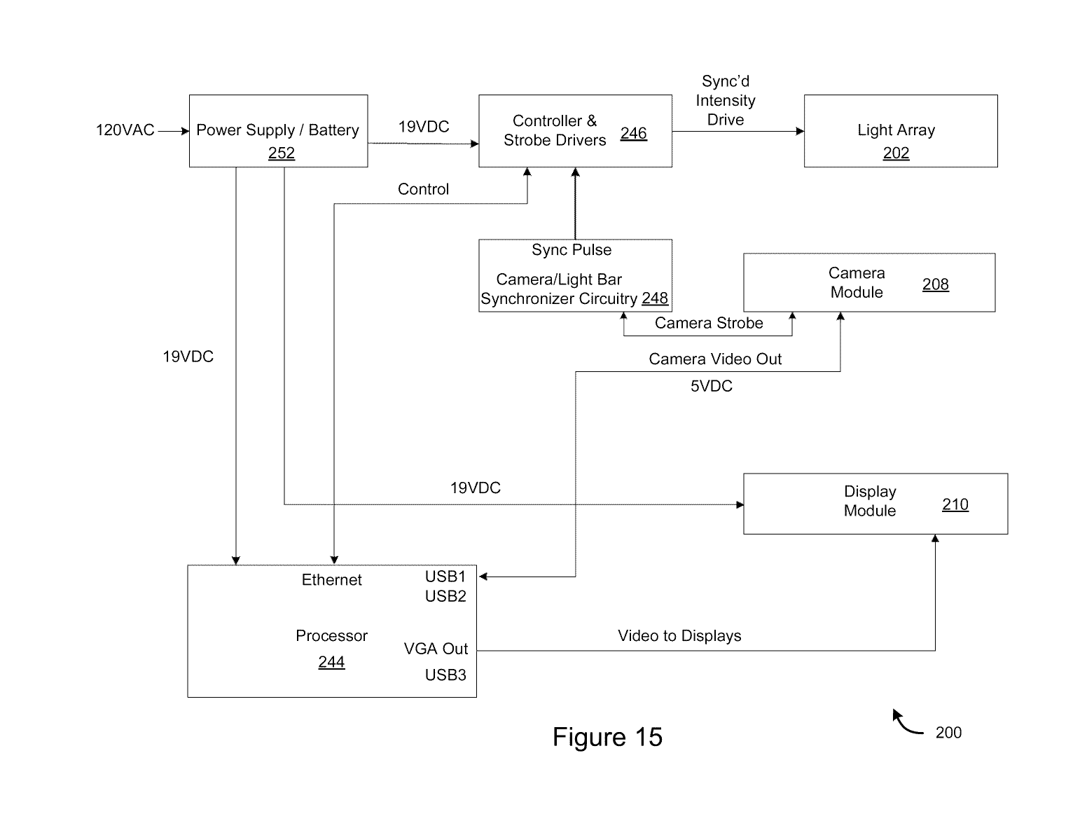

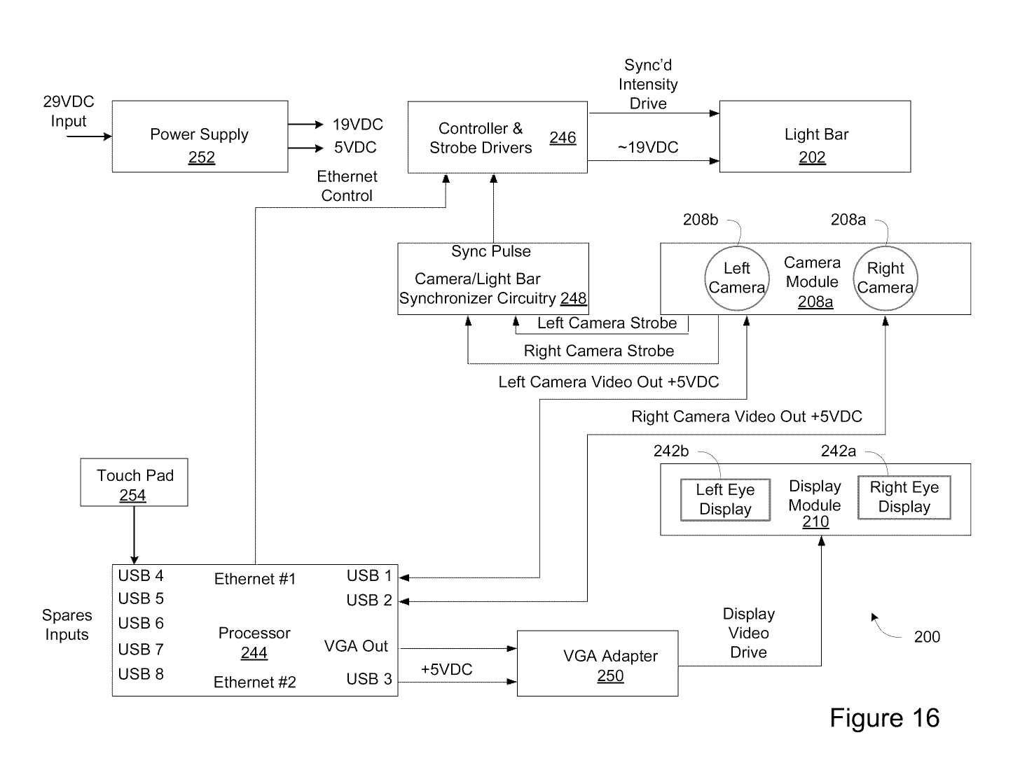

With reference to FIGS. 11-13, in some embodiments, the imaging system can be incorporated into a head mounted system. The system can be incorporated into eyewear 236 such as glasses (e.g., FIG. 11) or goggles, can be mounted to a headband 238 (e.g., FIG. 12) or visor, can be mounted to a helmet 240 (e.g., FIG. 13). The system can include a head mounted display 242, which can be positioned in front of an eye of the wearer so that the image of the target area can be presented to the eye of the wearer. Various display types can be used, such as a heads-up projection display, a reflection display, etc. In some embodiments, the image 212 can be displayed onto a monocle positioned in front of the wearer's eye. The head mounted display 242 can present different images to each eye. For example, one eye can be presented with an image of the veins while the other eye is presented with vital signs information, a GPS map, or a night vision image (e.g., generated using short wave infrared (SWIR) light). The head mounted system can have the light source 202 and the light sensor 208 (e.g., camera) mounted thereto, e.g., onto a temple portion of the headwear. In some embodiments, the light source can be located remotely (e.g., in a fanny pack or other wearable article), and the light can be directed from the remote light source to the head mounted system using a light guide (e.g., a fiber optic cable or bundle). This can prevent heat from the light source from heating the head mounted system, which can cause discomfort for the wearer.

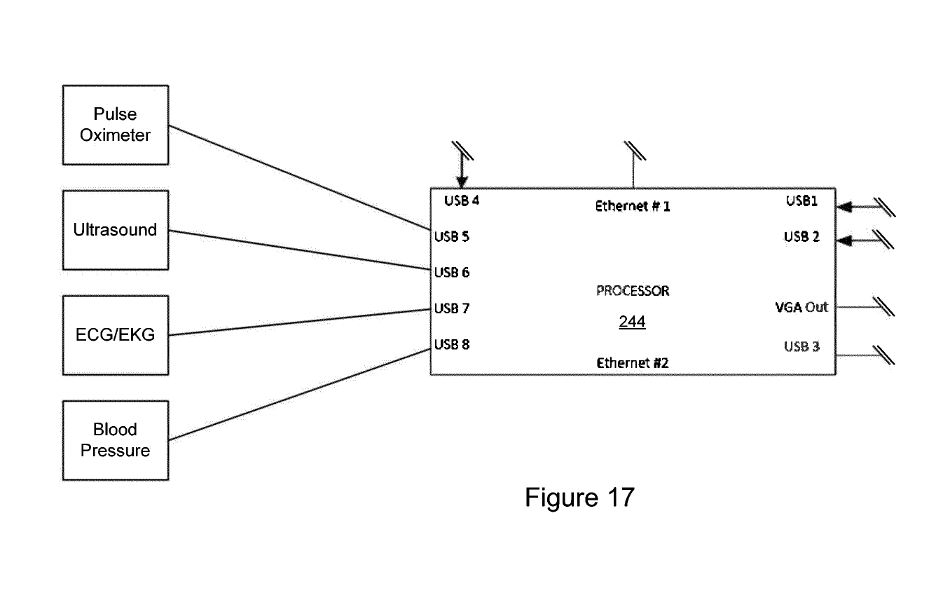

FIGS. 11-13 show a single camera 208 and a single head mounted display 242 disposed in front of one eye of the wearer. However, in some embodiments, as in FIG. 14, multiple cameras and/or multiple displays can be included. In some embodiments, the system can be configured to produce a stereoscopic 3D image. The system can include a first camera 208a, and a first display 242a that are associated with the right eye of the user. The system can include a second camera 208b and a second display 242b that are associated with the left eye of the user. The image generated by the first camera 208a can be presented on the first display 242a disposed to be viewed by the right eye, and the image generated by the second camera 208b can be presented on the second display 242b disposed to be viewed by the left eye. The first and second cameras 208a and 208b can be spaced apart so that the two images viewed by the wearer's eyes combine to provide a stereoscopic 3D image. FIG. 14 shows an example of a head mounted system (e.g., glasses) having two cameras 208a and 208b, one for the right eye and one for the left eye, which can provide a stereoscopic 3D image to the wearer (e.g., using two displays).

In some embodiments, the stereoscopic image in 3D can be presented on a single monitor or display device, which can be used with the devices shown in FIGS. 4-7B, or with another handheld or mobile device, or with other suitable devices. For example, the device shown in FIG. 4 can include two cameras that are spaced apart to produce right eye and left eye images, instead of the single camera 208 shown. The right eye and left eye images can be displayed on a single display device (e.g., on a handheld or mobile device), and eyewear (e.g., shuttering, polarizing, or color filtering) can be worn by the user to present the right eye image to the right eye and the left eye image to the left eye. In some embodiments, the display device can be configured to present a 3D image without the use of specialized eyewear, e.g., by using a parallax barrier or an lenticular array to direct the right eye image to the right eye and the left eye image to the left eye. In some embodiments, the system can provide a stereoscopic 3D NIR image to assist in determining patency and/or in evaluating infiltration or extravasation (e.g., by allowing the user to determine depth in the image).

In some embodiments, the one or more cameras 208a and 208b can be disposed at locations that are not in front of the wearer's eyes (e.g., not coincident with the normal line of sight of the eyes), such as on the temple or forehead portions of the eyewear (or other head mounted article (e.g., a helmet)). By locating the cameras at locations not in front of the wearer's eyes, the system can improve the wearer's viewing range of the surrounding environment, as compared to systems having the cameras 208a and 208b disposed in the wearer's line of sight. Also, by locating the cameras 208a and 208b at locations not in front of the wearer's eyes, the weight of the cameras 208a and 208b can be more centered, e.g., preventing the system from being front-heavy. Also, disposing the cameras 208a and 208b and/or light sources at locations not in front of the wearer's eyes can move the heat from the cameras 208a and 208b and/or light sources away from the wearer's face and/or can improve heat dissipation from the cameras and/or light sources. Disposing the cameras 208a and 208b at locations not in front of the wearer's eyes can also improve the aesthetic appearance of the system.

In some embodiments, the one or more cameras 208 can be located remote from the head mounted system, such as in a fanny pack or other wearable article. One or more light guides, e.g., a filter optic bundle, can direct the light that forms the image from the head mounted system to one or more remote light sensors 208. As discussed above, the light source 202 can also be located remote from the head mounted system and can be near the one or more light sensors 208 (e.g., located in the same fanny pack or other wearable article) so that the light guide(s) that transport light from the light source 202 to the head mounted system can run generally along the same path as the light guide(s) that transport the light from the head mounted system to the one or more light sensors 208. For example, in some embodiments, the light guides for the light source 202 and light sensor 208 can share a single covering or can be bound together, thereby reducing the number of cables that extend from the head mounted system.

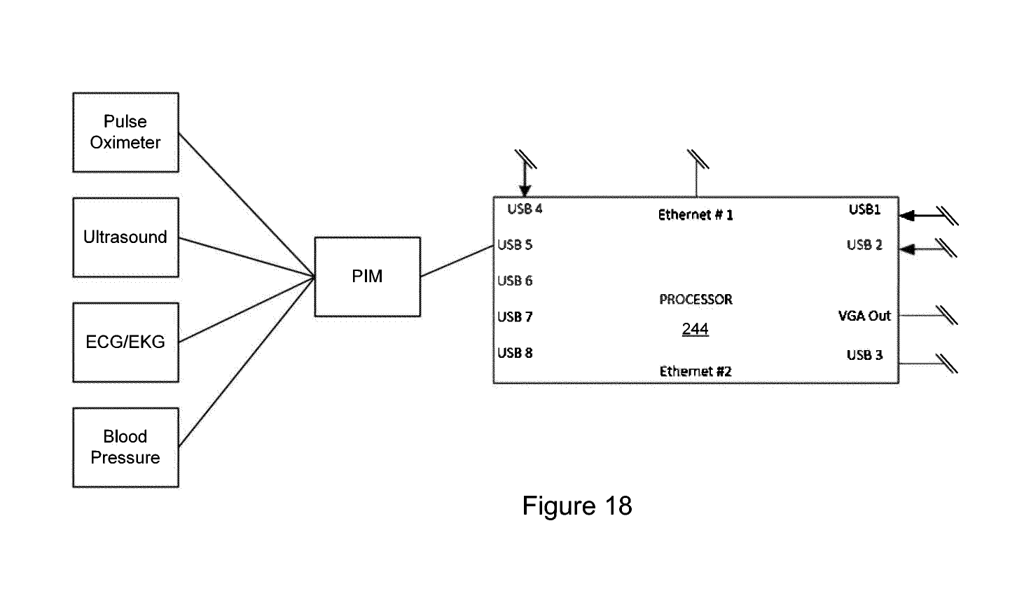

FIG. 15 is a block diagram of an example imaging system 200 according to certain embodiments disclosed herein. FIG. 16 is a block diagram of an example system that includes a right camera 208a, a left camera 208b, a right eye display 242a, and a left eye display 242b, which can produce a stereoscopic 3D image. The system can include a processor 244, which in some cases can be separate from the head mounted components and can be in communication with a camera module 208 and a display module 210 (e.g., via a cable or a wireless connection such as a Bluetooth communication link). The processor 244 can be configured to perform the operations described herein, or the processor 244 can be configured to execute computer code stored on a computer-readable memory module that causes the processor to perform the operations described herein. In some cases, the system 200 can include controller and strobe drivers 246, which can be instructions stored in computer-readable memory and/or can be circuitry or other electrical components configured to control the pulsing or sequencing of the light emitters of the light source 202 (e.g., the light bar or light array). In some embodiments, the system can include synchronizer circuitry 248 that can synchronize the light source 202 with the camera module 208, as discussed herein. The processor 244 can be in electronic communication with a display module 210 configured to display the image of the target area as discussed herein. In some embodiments a VGA adapter 250 (which can include a power converter) can be used to provide a signal to the display module 210.

The system can include a power supply 252 to provide power to the processor 244, to the light source 202, and to the various other components. The system can include an input device 254 (e.g., a touchpad) configured to receive input from the user, as discussed herein. Various other components can be included, for example a communication interface, which can be a wireless communication device, can be included for transferring information to and receiving information from other components, such as databases, remote systems, etc., as described in the various embodiments disclosed herein.

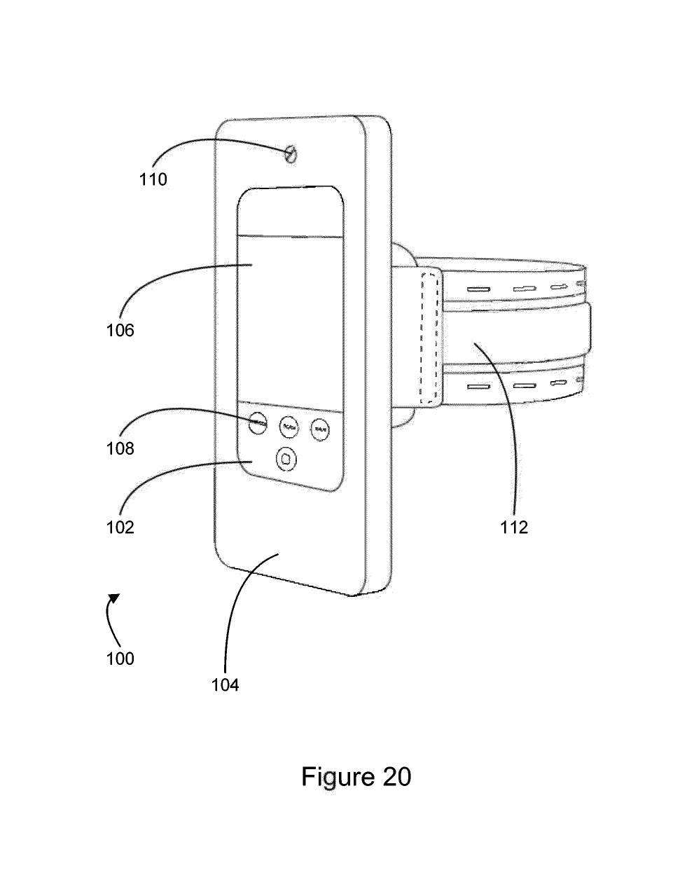

With reference to FIG. 17, in some embodiments, the imaging system 200 can be incorporated into a system that includes one or more additional medical components (some examples of which are shown in FIG. 17), such as components for measuring a patient's vitals (e.g., a pulse oximeter, an ultrasound device, an ECG/EKG, blood pressure monitor, a visual phonometry (VPM) (i.e., digital stethoscope), a thermometer, an otoscope, an exam camera, etc. The medical components can be configured to provide measurements as digital output, and the medical components can be configured to provide the digital measurements to the processor 244 through a cable (e.g., a USB connection) or through a wireless connection (e.g., a Bluetooth wireless communication link, a WiFi wireless communication link, a commercial communications radio link, or a military radio link, or combinations thereof).

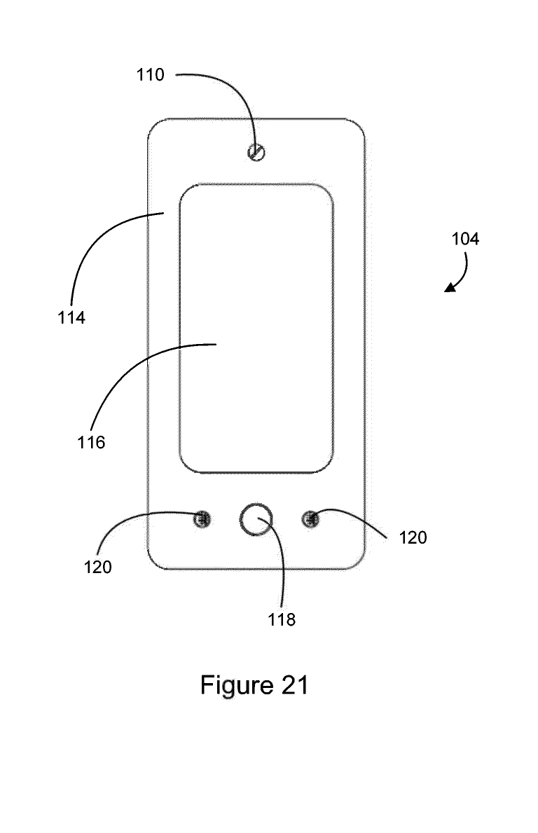

With reference to FIG. 18, in some embodiments, the medical components can connect to a Patient Integration Module (PIM) as a hub for some or all of the medical components, and the PIM can communicate with the Processor 244 via a cable or wirelessly. The PIM can receive a number of inputs cables from a number of medical components, and the PIM can couple to the imaging system (e.g., to the processor 244) by a single cable, or a smaller number of cables than the number of input cables from the medical components. The PIM may have an independent power supply (e.g., battery). The PIM can be a removable and/or disposable unit that stays with the patient for transport so only a single USB or wireless connection change is necessary to transfer the patient from one system to another. This disposable PIM has the added advantage of sanitation and infection control for patients as well as medical and transportation personnel. In some embodiments, the PIM can include a PIM processor and possibly a PIM display for displaying data from the medical components when the PIM is not in communication with the main processor and main display module.

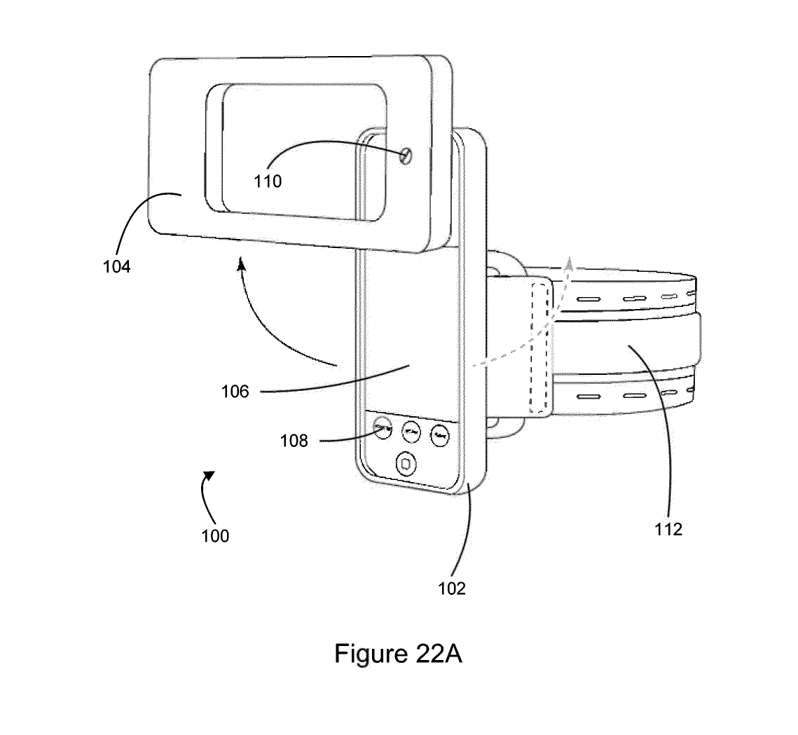

With reference to FIG. 19, in some embodiments, the system 200 can be configured to transmit data to a remote system 256, such as by a wireless connection or via a communication network. The remote system can be accessible to a doctor 258 or other medical practitioner. The system 200 can use NIR light for imaging veins as discussed herein. In some embodiments, the system can include a camera 260 for generating images using visible light. The system can send the visible light images to the remote system 256 for display so that the remote doctor 258 (or other medical practitioner) can observe the treatment of the patient. In some embodiments, the system 200 can include a camera 208 for producing images using non-visible light (e.g., NIR and/or SWIR light), which can be used for imaging a vein, as discussed herein. In some embodiments, the system 200 can be configured to produce night-vision images. Different light sensors can be used to produce the NIR images and the visible light images, or a single light sensor can be used to produce both images. In some embodiments, the system can be configured to produce images using short wave infrared (SWIR) light or using other types of light such as ultraviolet (UV) light. The different types of light sensors 208 and 260 can be incorporated into distinct camera modules that are mounted onto a single head mounted system 200, or the different types of light sensors 208 and 260 can share certain camera components and can be incorporated into a single camera module having multiple heads. In some embodiments, a light sensor can be used that is sensitive to NIR light and visible light so that a single light sensor can be used to produce the NIR images and the visible light images. For example, light sources and/or optical filters can be synchronized with the light sensor to produce multiple image types using a single sensor. In some embodiments, a twin camera may be used to produce one images for visible light and another image for non-visible light (e.g., NIR), and in some cases, the two images can be merged or interlaced.

In some embodiments, the system 200 can be configured to transfer the NIR images to the remote system 256 for display to the remote doctor 258. The system 200 can include additional medical components as discussed above, and the system 200 can be configured to transmit data collected using the additional medical components to the remote system 256 and to the remote doctor 258. In some embodiments, the information collected from the additional medical components can be displayed on the display 242 of the head mounted system.

In some embodiments, the system can include a two-way voice and/or data communication link to the remote system 256 to enable the remote doctor 258 to send instructions and other information to the user 262 performing the treatment on the patient. The instructions or other information received from the remote system 256 can be displayed on the display 242 of the head mounted system 200, or can be output to the user 262 by an audio device. Thus, the remote doctor 258 can oversee the treatment of the patient without the patient being transported to the doctor's location. This can result in faster treatment being delivered to the patient, reduced patient transportation costs, and reduced patient treatment costs because a patient can be treated on-site and released (e.g., without visiting the hospital). On-site treatment of a patient can sometimes be challenging because many treatments depend upon having an available infusion site (e.g., for delivering medication to a patient). However, it can be particularly challenging to establish an infusion site (e.g., by inserting an IV line) during on-site treatment because on-site treatment is often performed without the controlled environment that is present in a hospital or doctor's office. Accordingly, in some embodiments, it can be particularly advantageous to incorporate a system 200 for vein imaging into the wearable system of FIG. 19. The imaging system 200 can facilitate the insertion of an IV line and can make available many on-site treatment options that would not otherwise be readily available.

In some embodiments, a vein imaging system can be configured to be worn by a medical practitioner. For example, as discussed herein, one or more components of the vein imaging system can be worn on the head of the medical practitioner, for example, as eyewear. In some embodiments, one or more components of the vein imaging system can be worn on a forearm of the medical practitioner (e.g., using a strap as discussed herein). In some embodiments, one or more components of the vein imaging system can be incorporated into a pendant configured to be suspended on a lanyard or necklace worn on the neck of the medical practitioner (e.g., similar to an ID badge or stethoscope commonly worn by medical practitioners). The vein imaging system can be a handheld device (e.g., a smart phone or tablet or similar device) configured to be stored in a holster (e.g., worn on the hip of the medical practitioner).

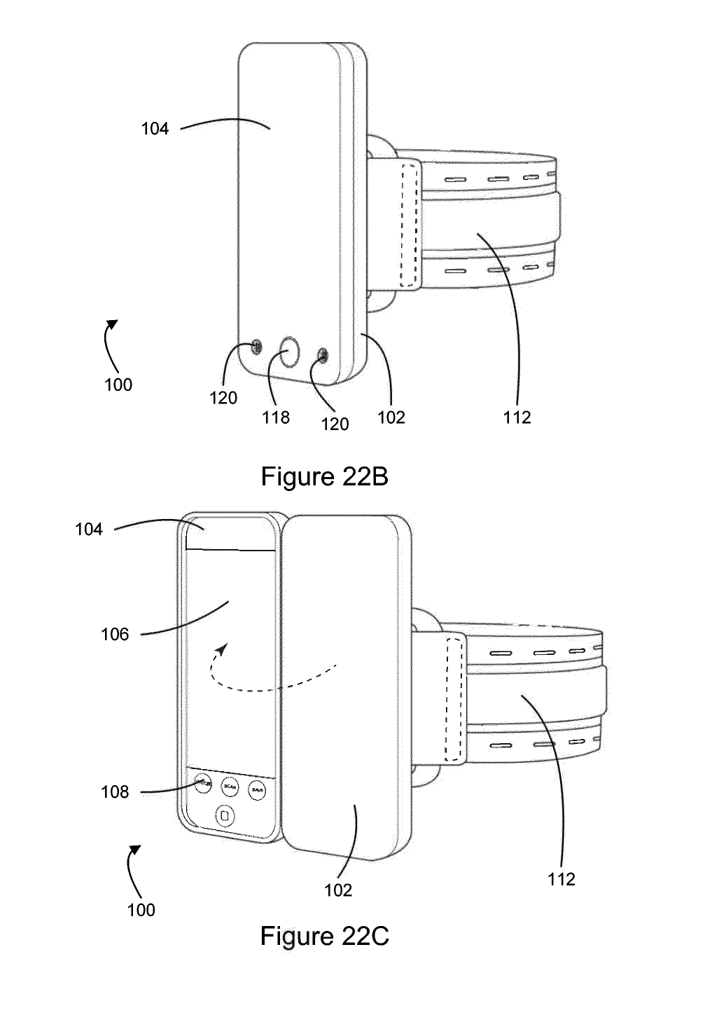

FIG. 20 shows an example embodiment of a vein imaging system 100 configured to be worn by a medical practitioner (e.g., on the forearm). The vein imaging system 100 can have features that are the same or similar to the other imaging systems disclosed herein, and the disclosure relating to other imaging systems can relate to the system 100 as well. The system 100 can include a main body 102 and a movable member 104. The main body 102 can include a display 106 and one or more user input elements 108 (e.g., buttons). In some embodiments, the display 106 can be a touch screen display configured to receive user input and some or all of the buttons 108 can be omitted. The main body 102 can be coupled to the movable member 104 at a connection point 110, which can be configured to allow the movable member 104 to move (e.g., pivot) relative to the main body 102. An engagement member, such as a strap 112, can be coupled to the system 100 (e.g., to the back of the main body 102), and the strap 112 can be configured to be worn on the forearm of the medical practitioner, or on any other suitable location of the medical practitioner. The strap 112 can use hook-and-loop fasteners (e.g., Velcro), or a clasp, or a lace, etc. to secure the strap 112 to the medical practitioner. In some embodiment, the system 100 can include one or more communication ports (e.g., a USB or other suitable port), which can be used to receive data from other devices, such as other medical devices as discussed herein. In some embodiments, the movable member 104 can have a notch on the side to allow a cable to access a communication port on the side of the main body 102.

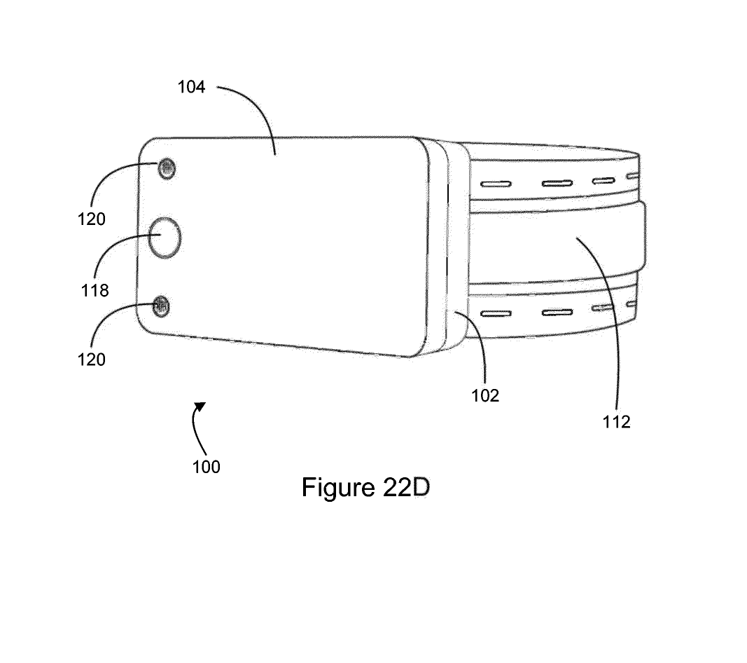

FIG. 21 shows the back side of the movable member 104 (which is hidden from view in FIG. 20), and the main body 102 is omitted from FIG. 21. The movable member 104 can include a connection point 110 configured to couple the movable member 104 to the main body 102. The movable member 104 can include a frame 114, and in some embodiments, the frame 114 can include an opening 116 configured to allow viewing of the display 106 when the movable member 104 is in the retracted position, as discussed below. The movable member 104 can include a camera 118, and one or more light sources 120 (e.g., NIR light sources as discussed herein). Additional components can be included, such as one or more optical filters (e.g., spectral filters, NIR filters, or polarizing filters), which are not specifically shown in FIG. 21, for simplicity. The camera 118 and/or the one or more light sources 120 can be positioned at generally the opposite side of the movable member 104 from the connection point 110, to facilitate the positioning of the camera 118 and/or the one or more light sources 120 over the area (e.g., on a patient) being imaged, as discussed below. For example, the camera 118 and/or the one or more light sources 120 can be positioned at least about 2 inches, at least about 3 inches, at least about 4 inches, at least about 5 inches, at least about 6 inches, or more away from the connection point 110. The camera 118 and/or the one or more light sources 120 can be positioned less than or equal to about 10 inches, less than or equal to about 8 inches, less than or equal to about 6 inches, less than or equal to about 4 inches, or less, from the connection point 110, to prevent the movable member 104 from being cumbersome (especially when in the extended position, as discussed below).

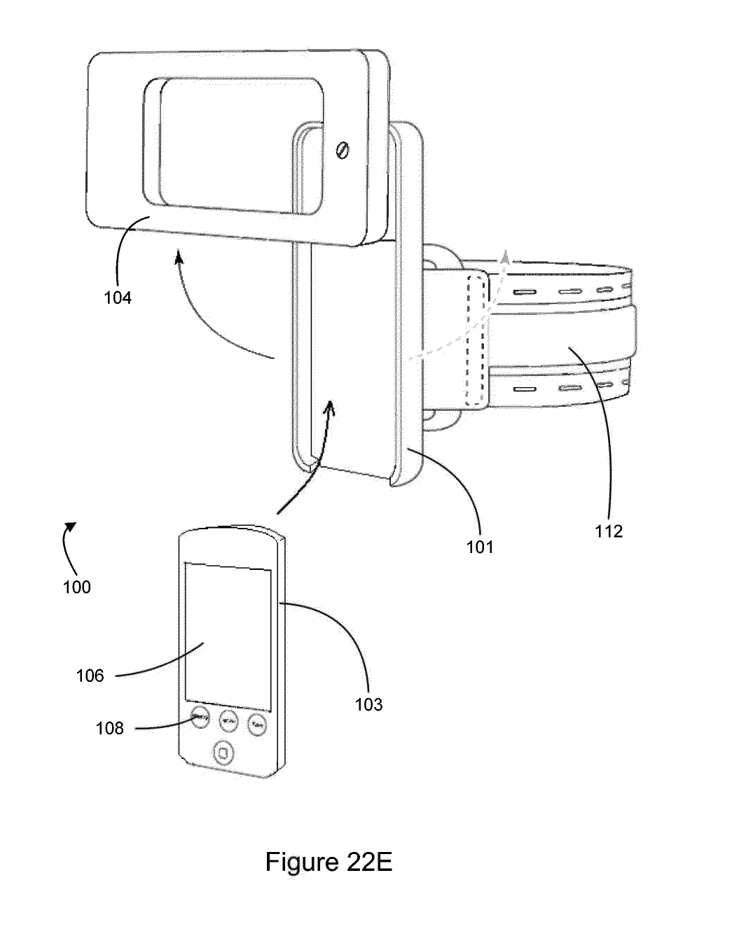

With reference to FIG. 22A, the movable member 104 can be configured to pivot about the connection point 110, which can include a rivet, a screw, a bolt, or other suitable mechanism that provides a pivot point. FIG. 20 shows the movable member 104 in a retracted or neutral position, and FIG. 22A shows the moveable member 104 in an extended or deployed position. In some embodiments, the camera 118 can be positioned over a cover (e.g., formed on or coupled to the main body) when in the retracted or neutral position, thereby protecting the camera when not in use. Although FIG. 22A shows the movable member 104 in an extended or deployed position that is pivoted in a clockwise direction, the movable member 104 can also be pivoted counter-clockwise to an extended position. In some embodiments, the movable member 104 can be configured to pivot at least about 45°, at least about 60°, at least about 75°, or about 90° between the retracted and extended positions. In some embodiments, the movable member 104 can be configured to rotate by less than or equal to about 135°, less than or equal to about 120°, less than or equal to about 105°, or about 90° between the retracted and extended positions. Although FIG. 22 shows the connection point 110 at the side opposite the one or more user input elements 108 (e.g., at the top), the connection point 110 can be at the opposite side than as shown in FIG. 22 (e.g., on the same side of the display 106 as the one or more user input elements 108, or the bottom).

Various alternatives are possible. For example, in some embodiments, the movable member can pivot at least about 135°, at least about 150°, or at least about 180° to the extended position. Thus, in some cases, if the system 100 is worn on the forearm of the medical practitioner, the camera 118 and/or the one or more light sources 120 can be positioned to the left or right of the wearer's arm, or near the wearer's hand when in use.

With reference to FIGS. 22B and 22C, in some embodiments, the connection point between the main body 102 and the movable member 104 can be a hinging connection point, and the movable member 104 can rotate (or flip) between the retracted (or neutral) and extended (or deployed) positions (e.g., as a clamshell configuration). In some embodiments, the hinge can be located at the top, or bottom, of the device so that the movable member 104 can rotate about an axis that is substantially transverse to the longitudinal axis of the device, or of the wearer's arm (e.g., to position the camera 118 near the hand of the wearer when the extended position). In some embodiments, as shown in FIGS. 22B and 22C, the hinge can be on the side, so that the movable member 104 rotates about an axis that is substantially parallel to the longitudinal axis of the device, or of the wearer's arm (e.g., to position the camera to the side of the wearer's arm when in the extended position). The clamshell configuration can cause the camera to be pointed upward when in the retracted (or closed) position (as shown in FIG. 22B) and can cause the camera to pointed downward when in the extended (or open) position (as shown in FIG. 22C). As illustrated in FIGS. 22B and 22C, the display 106 and/or the one or more inputs 108 can be positioned on the movable member 104 (e.g., on the opposite side as the camera 118 and one or more light sources 120). Accordingly, in some embodiments, no transfer of information is required between the main body 102 and the movable member 104. In some embodiments, the main body 102 can be smaller than shown and can merely attach the movable member 104 to the strap 112 (or other engagement member). Alternatively, the display 106 and/or the one or more inputs 108 can be positioned on the main body 102, e.g., such that they are uncovered when the movable member 104 is in the extended or deployed position.

Other alternatives are possible. For example, with reference to FIG. 22D in some embodiments, the movable member 104 can rotate about a hinge connection (similar to the clamshell configuration) as shown, for example, in FIGS. 22B and 22C, and the movable member 104 can also pivot (e.g., similar to FIG. 22A) with respect to the strap 112 (or other engagement member) and the wearer's arm. In some embodiments, the main body 102 and the movable portion 104 can rotate together with respect to the engagement member (e.g., strap 112). Thus, the movable member 104 and main body 102 can be rotated from the retracted or neutral position (shown in FIG. 22B) to a rotated or intermediate position (shown in FIG. 22D). The movable member 104 can then be flipped open to the deployed or extended configuration (e.g., as shown in FIG. 22C, except that the movable member 104 and main body 102 would also be rotated by about 90 degrees to the orientation of FIG. 22D). To transition the system from the retracted or neutral position to the deployed or extended position, the movable member 104 can first be flipped open (e.g., to the position shown in FIG. 22C) and the movable member 104 and main body 102 can be rotated to the orientation of FIG. 22D once the movable member 104 is in the open position. In some embodiments, the camera 118 and/or one or more light sources 120 can be positioned at the end of an arm, which can be an articulated arm, or a flex arm, to facilitate positioning the camera 118 and/or the one or more light sources over the imaging area while the system is worn by the medical practitioner.

In some embodiments, the connection point 110 can be configured to prevent over rotation of the movable member 104 past the extended or deployed position. In some embodiments, the connection point 110 can be configured to bias the movable member 104 to the retracted (or neutral) and/or extended (or deployed) positions, such that a threshold force is needed to dislodge the movable member from the retracted (or neutral) and/or extended (or deployed) positions, and a force below the threshold force is sufficient to move the movable member when it is position between the retracted and extended positions. For example, the one or more detents or friction features can cause the movable member 104 to tend to remain in the retracted or neutral position and/or the extended or deployed position. In some embodiments, the movable member 104 can be configured to move axially towards the main body 102 when the movable member 104 is transitioned to the retracted position, such that the frame 114 surrounds at least a portion of the main body 102. When the movable member 104 is transitioned out of the retracted or neutral position, the user can lift the movable member 104 axially away from the main body 102 until the frame 114 clears the main body 102 and allows the movable member 104 to rotate towards the extended or deployed position. In some embodiments, the connection point 110 can be spring loaded, or otherwise configured such that the movable member 104 is biased towards the main body 102 (e.g., in the retracted position).

When in the retracted or neutral position (as shown in FIG. 20), the camera 118 and/or the one or more light sources 120 can be positioned in a location or configuration that is not designed for use. For example, if the system is worn on the forearm of a medical practitioner, the retracted or neutral position can cause the camera 118 and/or the one or more light sources 120 to be positioned generally over the arm of the medical practitioner. When in the extended or deployed position, the camera 118 and/or the one or more light sources 120 can be positioned in a location or configuration that is designed for use of the camera 118 and/or the one or more light sources 120 (e.g., for imaging veins in a patient's anatomy). As mentioned above, the camera 118 and/or the one or more light sources 120 can be positioned at a sufficient distance from the connection point 110 to enable the camera 118 and/or the one or more light sources 120 to extend past the side of the wearer's forearm so that the one or more light sources 120 can direct light onto the imaging area (e.g., on the patient), and so the light reflected (e.g, scattered) by the imaging area can be received by the camera 118, to produce an image of the imaging area.

To use the vein imaging system 100, the medical practitioner can toggle the movable member 104 to the extended or deployed position, hold his or her forearm over the imaging area (e.g., on the patient), and operate the device by the one or more user input elements 108 (or touch screen display 106). The display 106 can be configured to display the image captured by the camera 118, e.g., to display the patient's vasculature in the imaging area. The medical practitioner can use the vein imaging system 100 for locating a vein to facilitate introduction of an IV or syringe needle into the patient, for assessing the patency of a vein, for identifying infiltration or extravasation, etc.

The system 100 can be used in connection with or combined with other features described herein. For example, the vein imaging system 100 can include a communication link for transmitting or receiving information from external sources. For example, images (or other data) from the system 100 can be transmitted to a remote system accessible by a different medical practitioner (e.g., a doctor), thereby enabling the doctor to oversee or review certain aspects of the patient's treatment or care from a remote location, similar to the discussion associated with FIG. 19. The system 100 can be configured to receive data (e.g., via a USB or other suitable connection) from other medical devices (e.g., a digital stethoscope, ultrasound device, pulse oximeter or, blood pressure monitor, etc.), as discussed in connection with at least FIGS. 17 and 18, and the system 100 can transfer or store information received from the other medical devices. In some embodiments, the system 100 can be configured to communicate with a database (e.g., electronic medical records (EMR)) for storing images and/or other data along with metadata for documenting a patient's treatment or care, as discussed above in connection with FIGS. 8 and 9.

In some embodiments, the main body 102 can have an integrated housing that includes the connection point 110 and also houses the display 106 and/or other elements of the main body 102 (e.g., as shown in FIG. 20-22A). In some embodiments, the movable member can have the display 106 and inputs 108, etc. (e.g., as shown in FIGS. 22B-22D). With reference to FIG. 22E, in some embodiments, the main body 102 can include an attachment portion 101 that includes the connection point 110, and is configured to receive a secondary housing portion 103 that houses the display 106 and/or other elements of the main body 102. In some embodiments, the attachment portion 101 can be a holster, such as a sled designed to slidably engage the secondary housing portion 103 to secure the secondary housing portion 103 to the attachment portion 101. The medical practitioner can thereby disengage the secondary housing portion 103 (including the display 106) from the attachment portion 101 and movable member 104. In some embodiments, the secondary housing can also house a processor. In some embodiments, a mobile device (e.g., a smart phone or tablet) can be used as the secondary housing 103 and associated components. The attachment portion 101 can have a communication interface element that is configured to engage a corresponding communication interface element on the secondary housing 103 to transfer information (e.g., commands from the user and images generated by the camera 118) between the secondary housing 103 (and associated components) and the movable member 104.

With reference to FIG. 22F, in some embodiments, the system does not include a separate main body and movable member. The camera and one or more light sources (hidden from view in FIG. 22F) can be incorporated onto the movable member 104 (e.g., onto the back thereof). A strap can mount the movable member 104 onto the medical practitioner (e.g., on to a forearm). A coupling mechanism can couple the movable member 104 to the strap 112 (or other engagement member). The coupling mechanism can be configured to allow the movable member 104 to rotate relative to the strap 112 (e.g., in a manner similar to the rotation of the movable member 104 with respect to the main body 102 discussed herein). The coupling mechanism can engage the movable member 104 at a location that is offset from the center so that, when the movable member 104 is pivoted (e.g., by about) 90° to the extended position, the camera and/or one or more light sources can be positioned clear of the wearer's arm, to enable the imaging system to image an imaging area below the wearer's arm. For example, the camera and/or the one or more light sources can be positioned at least about 2 inches, at least about 3 inches, at least about 4 inches, at least about 5 inches, at least about 6 inches, or more away from the pivot point. The camera and/or the one or more light sources can be positioned less than or equal to about 8 inches, less than or equal to about 6 inches, less than or equal to about 4 inches, or less, from the pivot point.

Various embodiments disclosed herein can be used to identify even low levels of infiltration or extravasation. For example, various embodiments can be configured to identify infiltration or extravasation as low as about 15 mL or less, about 10 mL or less, about 5 mL or less, about 3 mL or less, or about 1 mL or less, or about 0.5 mL. Various embodiments disclosed herein can be configured to identify infiltration and extravasation from veins that are generally about 3 mm to about 5 mm deep in the tissue of the patient. In some embodiments disclosed herein, the imaging systems can be configured to image veins and/or image extravasation or infiltration that is at least about 0.1 mm deep, at least about 1 mm deep, at least about 3 mm deep, at least about 5 mm deep, at least about 7 mm deep, or about 10 mm deep in the tissue.

In many hospital settings, a fluid is infused into a patent using an infusion pump (e.g., via an IV). In some circumstances, a patient's vein can become compromised which can cause the infused fluid to leak from the vein. In some cases, the infusion pump can continue to infuse fluid into the compromised vein thereby increasing the amount of extravasation or infiltration, which can cause serious injury or death. In some instances, an infusion pump can be configured to stop infusing fluid based on a change in pressure detected in the fluid line. For example, if a vein collapse can cause back pressure in the fluid line, which can be detected and used to stop the infusion pump. Also, a leakage in the vein can sometimes result in reduced pressure, which can also be detected and used to stop the infusion pump. Also, some systems can identify leakage based on changes in flow rate. These pressure- and flow-based techniques can identify infiltration or extravasation that results a sufficient pressure or flow change. Since movement of the patient, etc. can cause changes in the pressure in the line, or in the flow rate, the use of these pressure- and flow-based techniques can sometimes result in false alarms and/or undetected leakage. Also, some systems can use radio frequency (RF) technology to detect relatively large volumes of infiltration and extravasation.

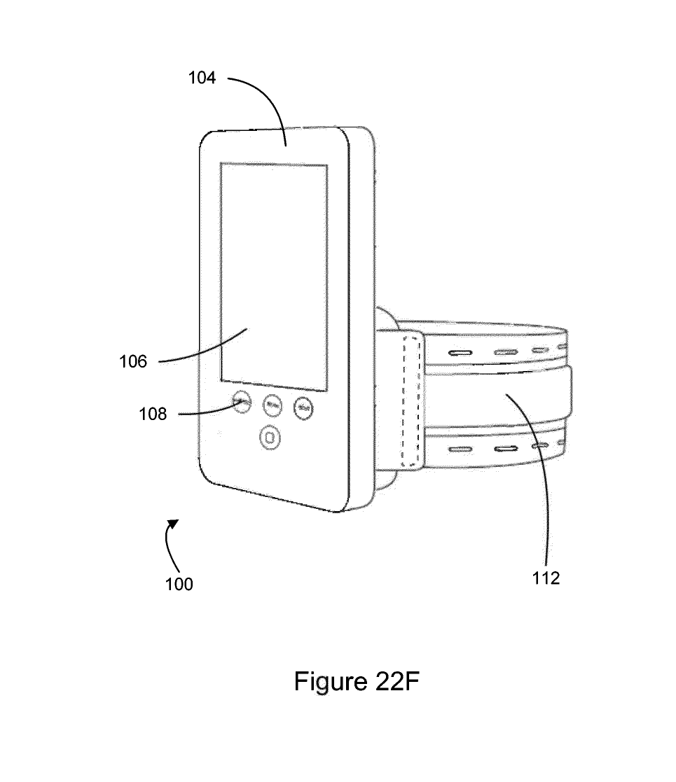

In some embodiments, a vein imaging system can be used to identify infiltration or extravasation (or otherwise determine that a vein's patency has been compromised) and the vein imaging system can be configured to cause an infusion pump associated with the compromised vein to automatically stop infusion and/or notify a medical practitioner. With reference to FIG. 23, an imaging head can be positioned relative to an infusion site to enable the imaging head to monitor the infusion site. The imaging head can include at least one light source and a camera (similar to the other embodiments discussed herein). The imaging head can be suspended over the infusion site (e.g., by a support member) so that the light source is configured to direct light (e.g., NIR light) onto the infusion site, and so that the camera is configured to receive light that is reflected (e.g., scattered) by the infusion site. In some embodiments, the imaging head can be positioned substantially over the infusion site. In some embodiments, the imaging head can be positioned to the side of the infusion site, and the imaging head can be angled towards the infusion site to enable the camera to obtain images of the infusion site. This configuration can provide the advantage of enabling the medical practitioner to see the infusion site and access the infusion site without manipulation of the imaging head.

In some embodiments, the support member can be configured to position the camera at a location that is a space away from the infusion site by a distance of at least about 0.5 inches, at least about 1.0 inches, or at least about 1.5 inches. In some embodiments, the camera can be positioned at a distance less than or equal to about 5 inches, less than or equal to about 3 inches, or less than or equal to about 2 inches from the infusion site. In some embodiments the camera can be configured to image an imaging area (e.g., associated with the infusion site) that is less than or equal to about 5 square inches, less than or equal to about 3 square inches, less than or equal to about 1 square inch, less than or equal to about 0.5 square inches, or less than or equal to about 0.1 square inches.

The camera can produce an image of the infusion site in which the veins are visible (e.g., as dark areas) and in which infiltration and extravasation are visible (e.g., as dark areas), as discussed herein. The imaging head can be configured to monitor the infusion site on a continuous or substantially continuous basis, or on a periodic (regular or irregular) basis (e.g., at least once per second, at least once per 5 seconds, once per 10 seconds, once per 30 seconds, once per minute, once per 5 minutes, etc.).

The imaging head can include a communication link, which can provide communication between the imaging head and one or more external devices. In some embodiments, the communication link can send data (e.g., image data) to an external processor, which can be configured to analyze the image data to determine the status of the infusion site (e.g., to identify infiltration or extravasation). The processor can be incorporated into an external device that includes additional features, such as a display (e.g., a touchscreen), and/or user interface elements (e.g., buttons). In some embodiments, the user can provide input, e.g., regarding what action should be taken in the event that the infusion site is determined to be compromised. The user can provide input that controls operation of the imaging head or the processor. For example, the processor can cause control information to be sent to the communication link (e.g., to control the rate at which the imaging head captures images for monitoring the infusion site). The user can provide input to adjust settings regarding the image processing performed by the processor to identify infiltration or extravasation. In some embodiment, the processor can be incorporated into a device that includes additional features not shown in FIG. 23.

The processor can be configured to take one or more actions (e.g., automatically) in the event that infiltration or extravasation is detected. For example, a command can be sent to an infusion pump to stop infusion of fluid to the infusion site. An alarm (e.g., an audible alarm) can be triggered. In some embodiments, the processor can send a notice to a nurse station to alert the nurse that the infusion site may be been compromised. In some embodiments, the processor can save information in the EMR or in another suitable database. For example, the image data showing infiltration or extravasation can be stored, metadata associated with the patent identification, the time of the images, or the time of the infiltration, can be stored. In some embodiments, the processor can store image data and/or other data (e.g., metadata) for images in which no extravasation or infiltration was identified (which can be used as evidence that the infusion site had not been compromised).

In some embodiments, the processor can be incorporated into the imaging head that is positioned over the infusion site, or the processor can be imaging head and processor can be integrated into a single housing or a single device. Accordingly, the imaging head can send commands or other information to the EMR, alarm, infusion pump, or nurse station directly. In some embodiments, an external device (e.g., the infusion pump) can include the processor. The communication link of the imaging head can send information (e.g., image data) to the infusion pump directly, and the processor of the infusion pump can be configured to analyze the image data to identify infiltration or extravasation. If infiltration or extravasation is identified, the infusion pump can automatically stop infusion of fluid into the infusion site.

The processor can perform image processing to identify whether infiltration or extravasation is represented in the image. Since infiltration and extravasation are represented in the image as dark areas, the brightness or darkness of the image (or of at least a portion of the image) can be used to identify infiltration and extravasation. For example, an image can be compared to a baseline image (which can be set by a medical practitioner, or can be automatically and periodically (regularly or irregularly) set a new baseline image (e.g., every hour, or every date, etc.). If the image (or portion thereof) is sufficiently darker than the baseline image (or portion thereof), the processor can determine that infiltration or extravasation is present. In some embodiments, the processor can identify infiltration or extravasation based at least in part on the rate of change of the brightness/darkness of the image (or at least a portion of the image). For example, the current image can be compared to prior images in a running time window to analyze the rate of change in the brightness/darkness of the image. In some embodiments, the user can define the length of the running window of time and/or the rate of change (or ranges) that can trigger an identification of infiltration or extravasation. For example, the rate at which the infiltration or extravasation develops can depend on the rate of infusion provided by the infusion pump. The time window can be about 1 minute or less, thereby comparing only images generated in the last 1 minute to determine whether there is any infiltration or extravasation. A short time window (e.g., 1 minute) can be useful when the rate of infusion is high or the fluid being infused is a high risk fluid (e.g., chemotherapy drugs), which can be especially harmful if leaked from the veins. The time window can be 30 seconds or less, 1 minute or less, 5 minutes or less, 10 minutes or less, 15 minutes or less, at least about 30 seconds, at least about 1 minute, at least about 5 minutes, at least about 10 minutes, or at least about 15 minutes. In some embodiments, image processing can be performed to enhance the image to facilitate identification of infiltration or extravasation. For example, contrast enhancement techniques, edge sharpening techniques, noise reduction techniques, and gamma correction techniques, etc. can be applied to the image.

Various other embodiments disclosed herein can incorporate features that are the same as, or similar to, the automatic infiltration or extravasation detection discussed herein. For example, an imaging system that is used by a medical practitioner to periodically check the patency of a vein can perform image processing to perform an automated assessment of whether infiltration or extravasation is present. A processor can compare the current image to one or more baseline images, and can compare the brightness or darkness of the images (or at least a portion thereof) to assess whether infiltration or extravasation is present, or to assess the extent of infiltration or extravasation. Other image processing techniques can be used, as discussed herein. For example, if an imaging enhancement agent is infused into the infusion site, the automated image processing can perform analysis that is tailored to the imaging enhancement agent. For example, if the imaging enhancement agent is a fluorescent material that emits a light of a different wavelength than the light that is reflected or scattered by the body tissue, the imaging system can be configured to distinguish between the different wavelengths so that the image processing can recognize the portion of the image that relates to the infused fluid. In some embodiments, if the system detects that infiltration or extravasation is present, the system can notify the medical practitioner that infiltration or extravasation is likely present. Accordingly, in some embodiments, the automated infiltration or extravasation detection system can be used as a guide or a confirmation, and the final determination of whether infiltration or extravasation is present (e.g., and whether to replace the infusion site) is made by the medical practitioner.

Similar image processing techniques can be used to assess blood flow in a vein (e.g., during a patency check). As mentioned above, acceptable blood flow can be visualized by a relatively bright area, that corresponds to an infused fluid (e.g., saline), moving along the path of a vein after the infused fluid is introduced through the infusion site. A series of images can be processed to track the movement of the bright area to make an automated assessment of blood flow in a vein. A similar approach can be used if an imaging enhancement agent is infused into the infusion site and tracked using automated image processing.

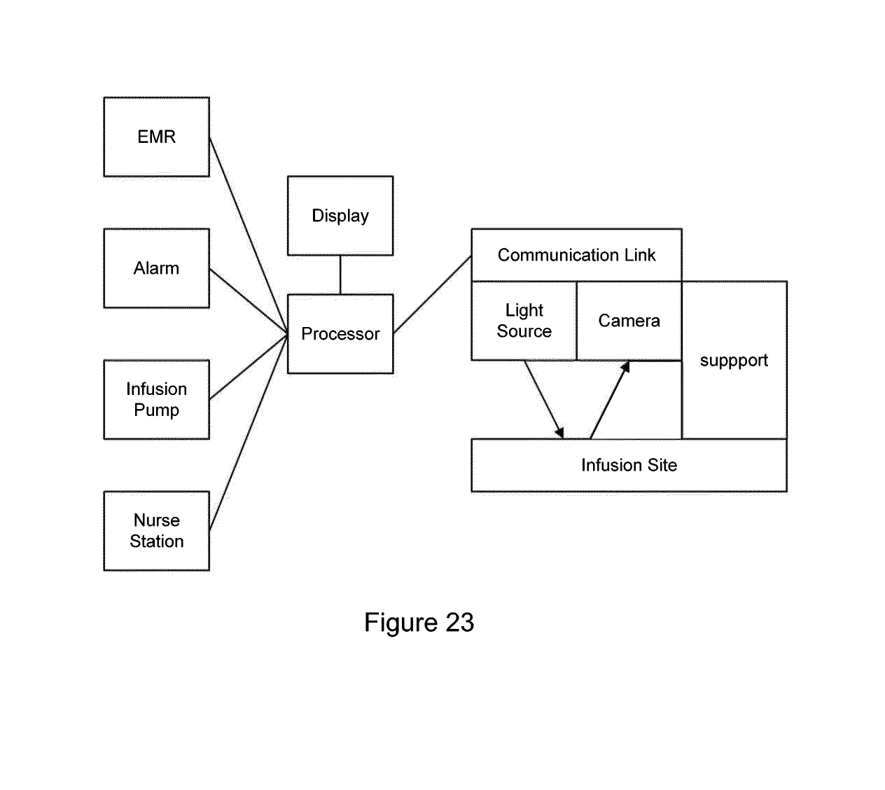

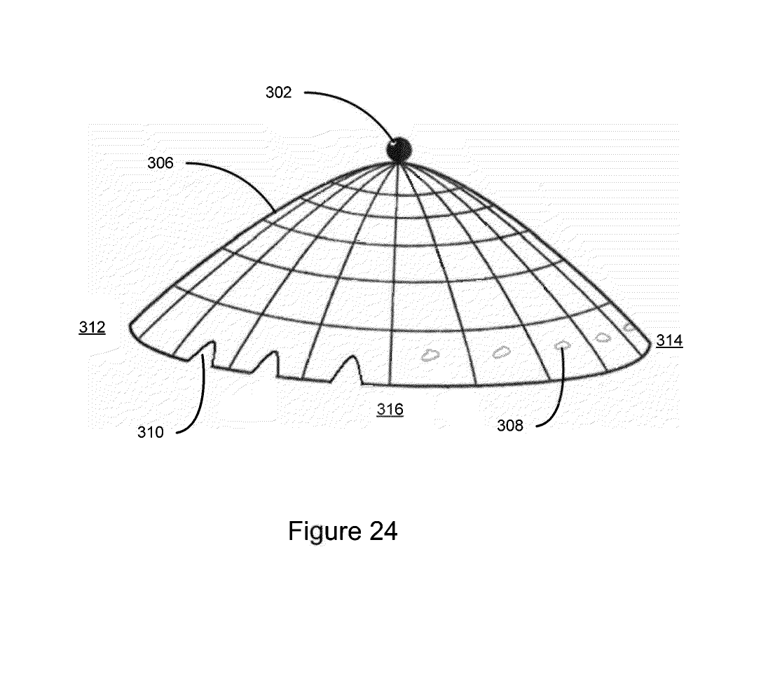

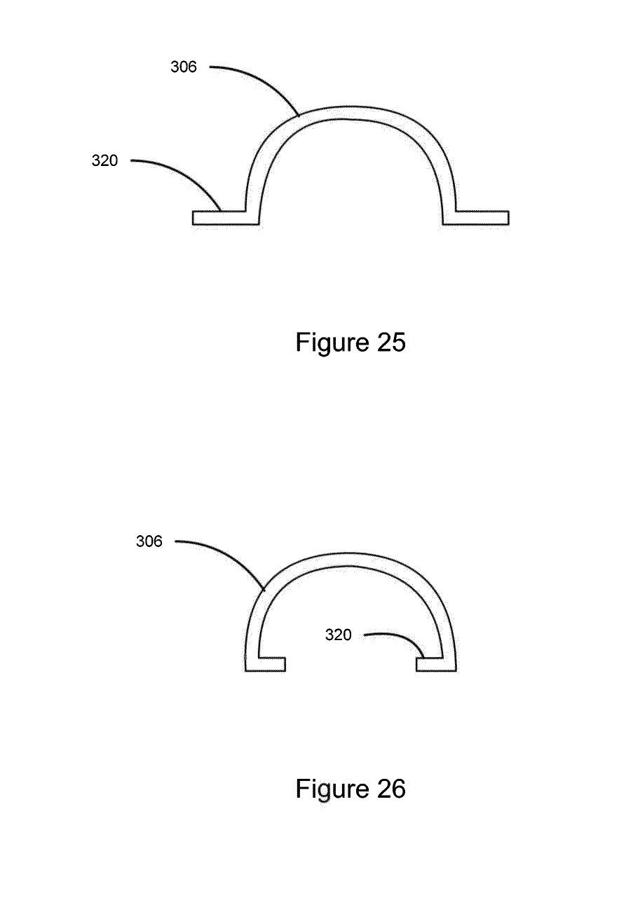

FIG. 24 shows an example embodiment of a support member 306 configured to position an imaging head 302 relative to the infusion site. The support member 306 can be a generally dome-shaped structure. The dome can be configured to suspend the imaging head 302 over the infusion site, to secure the IV line, and/or to protect the infusion site. The imaging head 302 can be positioned at the top portion (e.g., at or near the apex) of the dome 306. For example, the imaging head 302 can be mounted on the inside of the dome 306 using an adhesive, a molded bracket, hook-and-loop fasteners (Velcro), a screw, or other suitable attachment mechanism. In some embodiments, the light source can be used to generate an image to evaluate patency of a vein, as discussed herein. In some embodiments, the imaging head 302 can include one or more light sources (e.g., multiple light sources of different wavelengths) that are configured to be turned on for extended periods of time for treatment of the skin or tissue beneath the skin (e.g., about 3 mm to about 5 mm deep, or up to about 10 mm deep). In some embodiments, the dome structure 306 can be made of a clear material to allow the medical practitioner to see through the dome to the infusion site therein. In some embodiments, the dome 306 can include holes 308, which can be openings that are spaced apart from the edge of the dome 306, or notches 310, which can be openings that are disposed at the edge of the dome 306, that provide venting so that air can be exchanged between the infusion site and the surrounding area. Although FIG. 24 is shown as having both holes 308 and notches 310, some embodiments can include only holes 308, only notches 310, or a combination of holes 308 and notches 310. In some cases, the notches 310 can allow for a fluid line or a cable, etc. to enter the area under the dome 306 while allowing the fluid line or cable to remain adjacent to the skin of the patient. In some embodiments, a fluid line or cable can weave through a plurality of notches 310 to facilitate the securing of the line or cable to the dome 306. In some embodiments, the dome 306 can be a geodesic dome. The dome 306 can be of a generally circular shape, an oblong shape, a rectangular shape, etc. In some embodiments, a partial dome can be used (e.g., extending across about 180° of the circumference). In some embodiments, the dome 306 can be configured to fit onto or over various sized luer locks and IV tubing. In some embodiments, U-shaped pliable clamps (e.g., position as locations 312 and 314) can be used to fit various sizes of the extension set luer locks and IV tubing. The tube can be secured to the patient in various ways. For example a strap can be coupled to the dome (e.g., at location 316), and the strap can wrap around the arm, or other body portion, of the patient to position the dome 306 over the infusion site. In some embodiments, a biocompatible adhesive can be used to secure the dome 306 to the patient. As shown in FIGS. 25 and 26, the dome 306 can include a flange 320 at the base of the dome, and the bottom surface of the flange 320 can have the adhesive applied thereto so that it can be adhered to the patient. The flange 320 can extend out from the edge of the dome 306 (FIG. 25) or inwardly into the interior of the dome 306 (FIG. 26.) In some embodiments, the dome 306 can include a base portion and a movable upper portion, which can be moved to provide access to the infusion site. For example, a pivot member (e.g., a hinge) can be provided on a side of the dome 306, and the upper portion of the dome can pivot with respect to the base portion of the dome, to thereby open the dome, to enable the medical practitioner to access the infusion site without removing the dome. In some embodiments, the support member can be integrated with a structure configured to secure the IV at the infusion site.

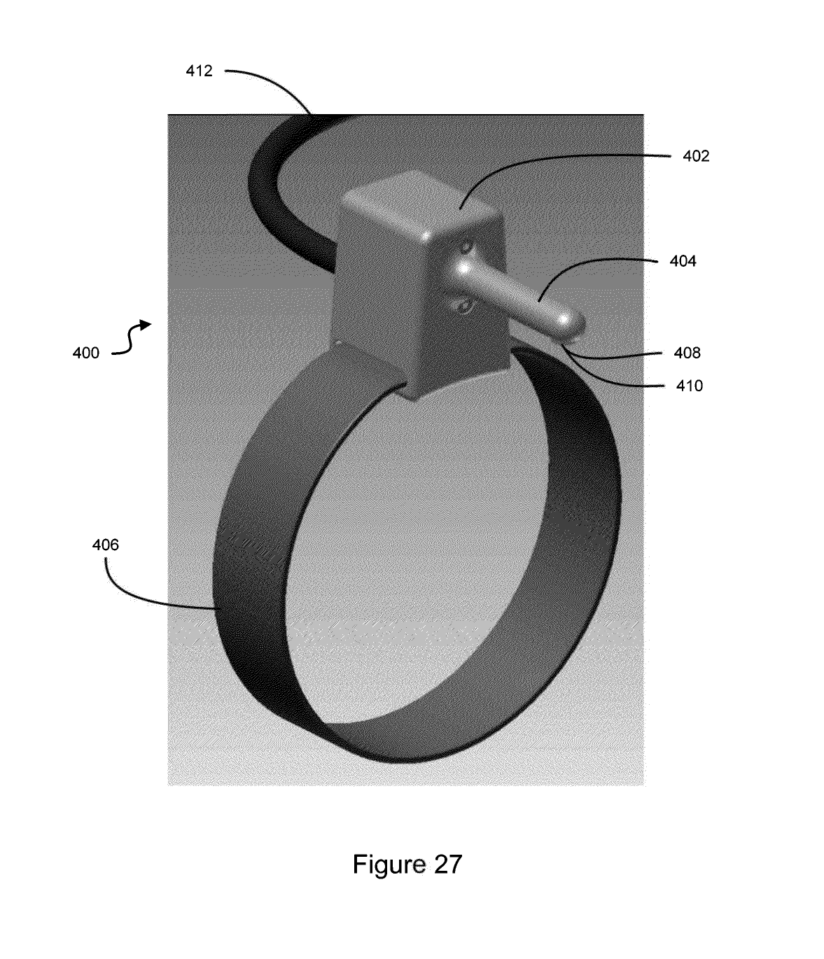

FIG. 27 shows an example embodiment of an imaging system 400, which can have features that are the same as, or similar to, the embodiments discussed in connection with FIG. 23-26. The system can be configured to automatically detect infiltration or extravasation as discussed herein. The system 400 can include a support member 406 (e.g., a strap) for positioning the system on a body portion of a patient, e.g., on a patient's arm or leg. The strap 406 can position a supporting portion generally adjacent to an infusion site or other target area to be imaged. The supporting portion 402 can, for example, have a slot that receives the strap 406 for coupling the supporting portion 402 to the strap 406. In some embodiments, a biocompatible adhesive can be used (either with or instead of the strap 406) to couple the system to the patient. For example the flat inch arch of the band can be adhered to the patient. An extension portion 404 can extend from the supporting portion 402 so that the extension portion 404 is positioned generally above the infusion site or other target area. The supporting portion 402 can have a height that suspends the extension portion 404 over the infusion site by a suitable amount, as discussed above. In some embodiments, a light source 408 and/or a light sensor 410 can be disposed on the extension portion 404 (e.g., at or near an end thereof) such that the light source 408 is suspended over the infusion site and such that light from the light source 408 is directed onto the target area, and such that the light sensor 410 is configured to receive light from the target area (e.g., light scattered or reflected therefrom). A cable 412 can provide power and/or instructions to the light source 408 and/or light sensor 410. Also, the cable 412 can transfer information from the sensor 410 to a controller, e.g., which can be configured to analyze image data to automatically detect infiltration or extravasation. In some embodiments, a controller can be included (e.g., inside the supporting portion 402) and the cable 412 can transfer information from the controller to other components (e.g., to shut off an infusion pump when infiltration or extravasation is detected. In some embodiments, the light source 408 and/or the light sensor 410 can be disposed in or on the supporting portion 402, and one or more light guides (e.g., fiber optic cables) can transfer the light from the light source to an output location on the extension portion 404, and the one or more light guides can transfer light receive by a input location on the extension portion 404.

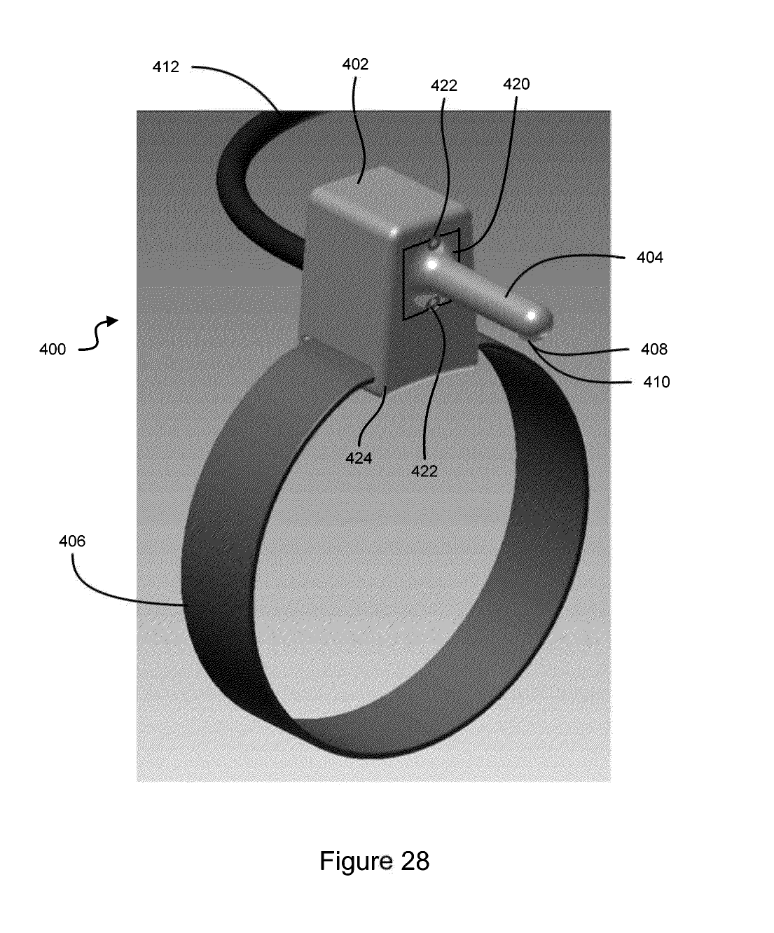

In some embodiments, at least some of the imaging system 400 can be disposable. With reference to FIG. 28, the system 400 can include an imaging head 420 that includes the light source 408 and light sensor 410, and the imaging head 420 can be removably coupled to the support member 406 (e.g., the strap). For example, the supporting portion 402 can have a disposable portion 424 that is configured to removably receive the imaging head 420. For example, coupling mechanisms 422 (e.g., screws, clamps, snaps, etc.) can couple the imaging head 420 to the disposable portion 424 of the supporting portion 402. Thus the strap 406 and the portion of the supporting portion 402 that contact the patient can be disposable, and the imaging head 420 that includes the light source 408 and light sensor 410 can be reusable. In some embodiments, the light source 408 and the light sensor 410 can be disposed inside the supporting portion 402, not in the extension portion 404. Accordingly, in some embodiments, the extension portion can be part of the disposable portion 424. Additional details and additional features that can be incorporated into the embodiments disclosed herein are provided in U.S. Pat. No. 5,519,208, titled INFRARED AIDED METHOD AND APPARATUS FOR VENOUS EXAMINATION, filed on Sep. 29, 1994 as U.S. patent application Ser. No. 08/315,128, which is hereby incorporated by reference in its entirety and made a part of this specification for all that it discloses; U.S. Pat. No. 5,608,210, titled INFRARED AIDED METHOD AND APPARATUS FOR VENOUS EXAMINATION, filed on Mar. 20, 1996 as U.S. patent application Ser. No. 08/618,744, which is hereby incorporated by reference in its entirety and made a part of this specification for all that it discloses; and U.S. Patent Application Publication No. 2008/0194930, titled INFRARED-VISIBLE NEEDLE, filed on Feb. 9, 2007 as U.S. patent application Ser. No. 11/673,326, which is hereby incorporated by reference in its entirety and made a part of this specification for all that it discloses.

The systems and methods disclosed herein can be implemented in hardware, software, firmware, or a combination thereof. Software can include computer-readable instructions stored in memory (e.g., non-transitory, tangible memory, such as solid state memory (e.g., ROM, EEPROM, FLASH, RAM), optical memory (e.g., a CD, DVD, Bluray disc, etc.), magnetic memory (e.g., a hard disc drive), etc.), configured to implement the algorithms on a general purpose computer, special purpose processors, or combinations thereof. For example, one or more computing devices, such as a processor, may execute program instructions stored in computer readable memory to carry out processes disclosed herein. Hardware may include state machines, one or more general purpose computers, and/or one or more special purpose processors. While certain types of user interfaces and controls are described herein for illustrative purposes, other types of user interfaces and controls may be used.

The embodiments discussed herein are provided by way of example, and various modifications can be made to the embodiments described herein. Certain features that are described in this disclosure in the context of separate embodiments can also be implemented in combination in a single embodiment. Conversely, various features that are described in the context of a single embodiment can be implemented in multiple embodiments separately or in various suitable subcombinations. Also, features described in connection with one combination can be excised from that combination and can be combined with other features in various combinations and subcombinations.

Similarly, while operations are depicted in the drawings or described in a particular order, the operations can be performed in a different order than shown or described. Other operations not depicted can be incorporated before, after, or simultaneously with the operations shown or described. In certain circumstances, parallel processing or multitasking can be used. Also, in some cases, the operations shown or discussed can be omitted or recombined to form various combinations and subcombinations.

1.-194. (canceled)

195. A method of detecting infiltration or extravasation in tissue surrounding an infusion site on a patient, the method comprising:
illuminating the tissue surrounding the infusion site with near infrared (NIR) light of a first wavelength during a first time;
receiving light of the first wavelength reflected or scattered from the tissue onto one or more light sensors;
illuminating the infusion site with near infrared (NIR) light of a second wavelength during a second time;
receiving light of the second wavelength reflected or scattered from the tissue onto the one or more light sensors;
generating one or more images of the tissue using the light of the first wavelength received by the one or more light sensor and using the light of the second wavelength received by the one or more light sensors;
displaying the one or more images of the tissue to a medical practitioner; and
detecting the presence of infiltration or extravasation in the tissue based at least in part on the one or more images of the tissue.

196. The method of claim 195, further comprising infusing an imaging enhancement agent through the infusion site, wherein detecting the presence of infiltration or extravasation in the tissue comprises identifying imaging enhancement agent that has leaked out of a vein.

197. The method of claim 196, wherein the imaging enhancement agent comprises at least one of a biocompatible dye, a biocompatible near infrared fluorescent material, and Indocyanine Green.

198. The method of claim 195, wherein the NIR light of the first wavelength and the NIR light of the second wavelength is absorbed by hemoglobin in blood such that the one or more images are configured to distinguish between blood and the tissue, and wherein detecting the presence of infiltration or extravasation in the tissue comprises identifying blood that has leaked out of a vein.

199. The method of claim 195, wherein detecting the presence of infiltration or extravasation comprises performing image processing on the one or more images using a computer processor to detect the presence of infiltration or extravasation.

200. The method of claim 195, further comprising:
associating the one or more images with a patient identifier and with time information; and
storing the one or more images, the associated patient identifier, and the associated time information in a patient treatment archive in a computer-readable memory device.

201. The method of claim 200, further comprising:
receiving a notification of a claim of medical error for the patient; and
retrieving, using one or more computer processors in communication with the computer-readable memory device, the one or more images of the tissue surrounding the infusion site on the patient from the patient treatment archive.

202. The method of claim 195, wherein receiving the light of the first wavelength onto the one or more light sensors comprises receiving the light of the first wavelength onto a first light sensor; and wherein receiving the light of the second wavelength onto the one or more light sensors comprises receiving the light of the second wavelength onto a second light sensor.

203. The method of claim 195, wherein generating one or more images comprises:
generating a first image of the tissue using the light of the first wavelength received by the one or more light sensors; and
generating a second image of the tissue using the light of the second wavelength received by the one or more light sensors; and
wherein displaying the one or more image comprises displaying the first image and the second image in rapid succession so that the first image and the second image merge when viewed by the medical practitioner.

204. The method of claim 195, wherein generating one or more images comprises generating a composite image by combining image data from the light of the first wavelength and image data from the light of the second wavelength.

205. A system for facilitating the detection of infiltration or extravasation in a target area at an infusion site on a patient, the system comprising:
one or more light sources configured to pulse on and off;
one or more light sensors configured to receive light from the one or more light sources that is reflected or scattered by the target area;
a controller configured to generate one or more images of the target area from the light received by the one or more light sensors;
a display configured to display the one or more images of the target area;
wherein the system is configured such that the displayed one or more images indicate the presence of infiltration or extravasation when infiltration or extravasation is present in the target area.

206. The system of claim 205, wherein the one or more light sources are configured to emit light that is configured to be absorbed by hemoglobin such that the one or more images are configured to distinguish between hemoglobin in blood and surrounding tissue, and wherein the indication of the presence of infiltration or extravasation in the one or more images comprises an image of blood that has leaked out of a vein.

207. The system of claim 205, further comprising an infusion device containing an imaging enhancement agent, wherein the infusing device is configured to infuse the imaging enhancement agent into the infusion site.

208. The system of claim 207, wherein the imaging enhancement agent comprises at least one of a biocompatible dye, a biocompatible near infrared fluorescent material, and Indocyanine Green.

209. The system of claim 205, wherein the controller is configured to:
analyze the one or more images to determine whether infiltration or extravasation is likely present in the target area based at least in part on the one or more images; and
display an indication on the display of whether infiltration or extravasation is likely present in the target area.

210. The system of claim 205, further comprising a documentation system configured to:
associate the one or more images with a patient identifier and with time information; and
store the one or more images, the associated patient identifier, and the associated time information in a patient treatment archive.

211. The system of claim 205, wherein the one or more light sources are configured to emit near infrared (NIR) light.

212. The system of claim 205, further comprising headwear, wherein the one or more light sources and the one or more light sensors are on the headwear, and wherein the display comprises a head mountable display system.

213. The system of claim 205, wherein the one or more light sources comprise:
a first light source configured to emit light of a first wavelength; and
a second light source configured to emit light of a second wavelength;
wherein the controller is configured to sequentially pulse the first light source and the second light source at a rate corresponding to an imaging rate of the one or more light sensors.

214. A system for facilitating the detection of infiltration or extravasation in a target area at an infusion site on a patient, the system comprising:
a light source configured to direct light onto the target area;
a light sensor configured to receive light from the target area;
a controller configured to generate an image of the target area from the light received by the light sensor;
a display configured to display the image of the target area;
wherein the system is configured such that the displayed image indicates the presence of infiltration or extravasation when infiltration or extravasation is present in the target area.

215. The system of claim 214, wherein the light source is configured to emit light that is configured to be absorbed by hemoglobin such that the image is configured to distinguish between hemoglobin in blood and surrounding tissue, and wherein the indication of the presence of infiltration or extravasation in the image comprises an image of blood that has leaked out of a vein.

216. The system of claim 214, further comprising an infusion device containing an imaging enhancement agent, wherein the infusing device is configured to infuse the imaging enhancement agent into the infusion site.

217. The system of claim 214, further comprising a documentation system configured to:
associate the image with a patient identifier and with time information; and
store the image, the associated patient identifier, and the associated time information in a patient treatment archive.

218. The system of claim 214, further comprising headwear, wherein the light source and the light sensor are on the headwear, and wherein the display comprises a head mountable display system.
